(19) United States
(12) Patent Application Publication
(10) Pub. No.: US 2007/0077235 A1
(43) Pub. Date: Apr. 5, 2007

(54) Composition and method of treating mastitis

(76) Inventors: Lawrence Loomis, Columbia, MD (US); Vincent Fischetti, West Hempstead, NY (US)

Correspondence Address: Jonathan Edwin Grant, 2107 Hounds Run Place, Silver Spring, MD, 20906, US

(21) Appl. No.: 11/510,805

(22) Filed: Aug. 14, 2006

Related U.S. Application Data
(63) Continuation of application No. PCT/US05/04048, filed on Feb. 10, 2005.
(63) Continuation of application No. PCT/US04/01077, filed on Jan. 16, 2004.
(60) Provisional application No. 60/543,651, filed on Feb. 12, 2004.

(51) Int. Cl. A61K 35/76

(52) U.S. Cl. 424/93.6

(57) ABSTRACT

A composition and method for treating bacterial infections by the use of an effective amount of at least one lytic specific for the bacteria causing specific. The lytic enzyme is genetically coded for by a bacteriophage which may be specific for said bacteria. The enzyme may be at least one lytic protein or peptides in a natural or modified form.

This provisional application incorporates U.S. provisional application No. 60/440,352, and PCT/US 04/01077 filed on Jan. 16, 2004 the entirety of which is hereby incorporated by reference.

### BACKGROUND

1. Field of the Disclosure

The disclosure relates to methods and compositions for treating bacterial infections with bacteria-associated phage proteins, enzymes or peptides, and/or peptide fragments thereof. More specifically, the disclosure pertains to phage lytic and/or holin proteins, or peptides and peptide fragments thereof, blended with a carrier for the treatment and prophylaxis of bacterial infections for mastitis.

2. Description of the Prior Art

A major problem in medicine has been the development of drug resistant bacteria as more antibiotics are used for a wide variety of illnesses and other conditions. The over utilization of antibiotics has increased the number of bacteria showing resistance. Furthermore, broadly reactive antibiotics can effect normal flora and can cause antibiotic resistance in these organisms because of the frequency of drug use. The number of people becoming hyper allogenic to antibiotics appears to be increasing because of antibiotic overutilization. Accordingly, there is a commercial need for new antibiotics (or bacterial killing substances), especially those that operate in new modalities or provide new means to kill pathogenic bacteria.

Attempts have been made to treat bacterial diseases through the use of bacteriophages.

U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses a procedure of improved dental hygiene which comprises introducing into the mouth bacteriophages parasitic to bacteria which possess the property of readily adhering to the salivary pellicle.

However, the direct introduction of bacteriophages to prevent or fight diseases has certain drawbacks. Specifically, both the bacteria and the phage have to be in the correct and synchronized growth cycles for the phage to attach to the bacteria. Additionally, there must be the right number of phages to attach to the bacteria; if there are too many or too few phages, there will be no attachment and no production of the lysing enzyme. The phage must also be sufficiently active. Phages are inhibited by bacterial debris from the invading organism which block the phage's attachment site to its receptor. Further complicating the direct use of a bacteriophage to treat bacterial infections is the possibility of immunological reactions, rendering the phage non-functional. Most importantly, the targeted bacteria mutates its surface receptors for the bacteriophage, rendering the bacteria non-infective.

Consequently, others have explored the use of safer and more effective means to treat and prevent bacterial infections. In particular, the use of phage associated lytic enzymes has been explored.

Bacteriophage lysins are a class of bacteriolytic agents recently proposed for eradicating the nasopharyngeal carriage of pathogenic streptococci. (Loeffler, J. M., Nelson, D. & Fischetti, V. A. Rapid killing of Streptococcus pneumoniae with a bacteriophage cell wall hydrolase. Science 294, 2170-2 (2001); Nelson, D., Loomis, L. & Fischetti, V. A. Prevention and elimination of upper respiratory colonization of mice by group A streptococci by using a bacteriophage lytic enzyme. Proc Natl Acad Sci USA 98, 4107-12 (2001)). Lysins are part of the lytic mechanism used by double stranded DNA (dsDNA) phage to coordinate host lysis with completion of viral assembly. Wang, I. N., Smith, D. L. & Young, R. Holins: the protein clocks of bacteriophage infections. Annu Rev Microbiol 54, 799-825 (2000). Late in infection, lysin translocates into the cell wall matrix where it rapidly hydrolyzes covalent bonds essential for peptidoglycan integrity, causing bacterial lysis and concomitant progeny phage release. Lysin family members exhibit a modular design in which a usually well conserved catalytic domain is fused to a more divergent specificity or binding domain. See, Lopez, R., Garcia, E., Garcia, P. & Garcia, J. L. The pneumococcal cell wall degrading enzymes: a modular design to create new lysins? Microb Drug Resist 3, 199-211 (1997); Loessner, M. J., Kramer, K., Ebel, F. & Scherer, S. C-terminal domains of Listeria monocytogenes bacteriophage murein hydrolases determine specific recognition and high-affinity binding to bacterial cell wall carbohydrates. Mol Microbiol 44, 335-49 (2002). In many cases a fragment having catalytic activity can be determined and is preferably linked to a binding site region. The linkage optionally may be made through a third joining piece. High affinity binding is directed towards species- or strain-specific cell wall carbohydrate ligands that are often essential for bacterial viability, thus implying that intrinsic lysin resistance will be rare or impossible.

U.S. Pat. No. 5,604,109 (Fischetti et al.) relates to the rapid detection of Group A streptococci in clinical specimens, through the enzymatic digestion of the bacterial cell wall by a semi-purified Group C streptococcal phage associated lysin enzyme. Embodiments of the disclosure are based upon the discovery that phage associated lytic enzymes specific for bacteria infected with a specific phage can effectively and efficiently break down the cell wall of the bacterium in question. At the same time, in most if not all cases, the semi purified enzyme is lacking mammalian cell receptors and therefore tends to be less destructive to mammalian proteins and tissues when present during the digestion of the bacterial cell wall.

U.S. Pat. No. 5,985,271 (Fischetti & Loomis), U.S. Pat. No. 5,997,862 (Fischetti & Loomis), and U.S. Pat. No. 6,017,528 (Fischetti & Loomis) disclose the compositions and their use in an oral delivery mode, such as a candy, chewing gum, lozenge, troche, tablet, a powder, an aerosol, a liquid or a liquid spray that contains a lysin enzyme produced by group C streptococcal bacteria infected with a C1 bacteriophage for the prophylactic and therapeutic treatment of Streptococcal A throat infections, commonly known as strep throat. This lysin enzyme is described in U.S. Pat. No. 5,604,109.

U.S. Pat. No.6,056,954 (Fischetti & Loomis) discloses a method and composition for the prophylactic and/or therapeutic treatment of bacterial infections that comprises the administeration of an effective amount of at least one lytic enzyme produced by a bacteria infected with a bacteriophage specific for the bacteria The lytic enzyme preferably comprises a carrier suitable for delivering the lytic enzyme to the site of the infection. This method and treatment may be used for treating and eliminating bacterial infestations anywhere, including upper respiratory infections, topical and systemic infections, vaginal infections, eye infections, ear infections, infections requiring parenteral treatment, as well as for the elimination of bacteria on any surface.

U.S. Pat. No. 6,056,955 (Fischetti and Loomis) discloses a method and composition for the topical treatment of streptococcal infections by the use of a lysin enzyme blended with a carrier suitable for topical application to dermal tissues. The method for the treatment of streptococcal infections describes the administration of a composition comprising an effective amount of a therapeutic agent, where the therapeutic agent is a lysin enzyme produced by group C streptococcal bacteria infected with a C1 bacteriophage. The therapeutic agent can be in a pharmaceutically acceptable carrier.

U.S. Pat. No. 6,248,324 (Fischetti and Loomis) discloses a method for treatment of bacterial infections of the digestive tract comprising the administration of a lytic enzyme specific for the infecting bacteria. The lytic enzyme is preferably in a carrier for delivering said lytic enzyme. The bacteria species to be treated is selected from the group consisting of Listeria, Salmonella, E. coli, Campylobacter, and combinations thereof. The carrier for delivering at least one lytic enzyme to the digestive tract is selected from the group consisting of suppositories, enemas, syrups, or enteric coated pills.

U.S. Pat. No. 6,254,866 (Fischetti and Loomis ) discloses a method for treating bacterial infections of the digestive tract comprising the administration of a lytic enzyme specific for the infecting bacteria. There is preferably a carrier for delivering the lytic enzyme to the site of the infection in the digestive tract. The bacteria to be treated is selected from the group consisting of Listeria, Salmonella, E. coli, Campylobacter, and combinations thereof. The carrier is selected from the group consisting of suppositories, enemas, syrups, or enteric coated pills.

U.S. Pat. No. 6,264,945 (Fischetti and Loomis) discloses a method and composition for the treatment of bacterial infections by the parenteral introduction of at least one phage associated lytic enzyme specific for the invasive bacteria and an appropriate carrier for delivering the lytic enzyme into a patient. The injection can be done intramuscularly, subcutaneously, or intravenously.

U.S. Pat. No. 6,238,661 (Fischetti and Loomis) discloses compositions and methods for the prophylactic and therapeutic treatment of bacterial infections which comprise administering to an individual an effective amount of a composition comprising an effective amount of lytic enzyme and a carrier for delivering the lytic enzyme. This method and composition can be used for the treatment of upper respiratory infections, skin infections, wounds, burns, vaginal infections, eye infections, intestinal disorders and dental problems.

PCT/US04/01077 (Loomis and Fischetti) discloses the compositions and methods for the prophylactic and therapeutic treatment of bacterial infections of some diseases in various animal species.

Embodiments of the disclosure are based upon the discovery that phage associated lytic enzymes specific for bacteria infected with a specific phage can effectively and efficiently break down the cell wall of the bacterium in question. At the same time, in most if not all cases, the semi or fully purified enzyme is lacking in mammalian cell receptors and therefore tends to be less destructive to mammalian proteins and tissues when present during the digestion of the bacterial cell wall.

The same general technique used to produce and purify a lysin enzyme shown in U.S. Pat. No. 5,604,109 may be used to manufacture other lytic enzymes produced by bacteria infected with a bacteriophage specific for that bacteria. Depending on the bacteria, there may be variations in the growth media and conditions.

The use of phage associated lytic enzymes produced by the infection of a bacteria with a bacteria specific phage has numerous advantages for the treatment of diseases. The lytic enzymes are targeted against a specific bacteria and these do not interfere with normal flora. Also, lytic enzymes primarily attack cell wall structures, which are not affected by plasmid variation. The actions of the lytic enzymes are fast and do not depend on bacterial growth. Additionally, lytic enzymes can be directed to the mucosal lining, where, in residence, they can kill colonizing bacteria.

### SUMMARY OF THE DISCLOSURE

The present disclosure discloses the use of bacteriophage associated lytic proteins and holin proteins for the treatment of mastitis. The phage associated lytic and holin proteins include their isozymes, analogs, and variants thereof in a natural or modified form either alone or in combination with complementary agents.

Accordingly, the present disclosure provides a pharmaceutical composition containing at least one bacteria-associated phage protein and peptides and peptide fragments thereof, isolated from one or more bacteria species, wherein the phage proteins and peptide fragments thereof include phage lytic and/or holin proteins. In one embodiment of the disclosure, the lytic and/or holin proteins, including their isozymes, analogs, or variants, are used in an altered form. In another embodiment of the disclosure, the lytic and/or holin proteins, including their isozymes, analogs, or variants, are used in a modified form or a combination of natural and modified forms. The altered forms of lytic and holin proteins are made synthetically by chemical synthesis and/or DNA recombinant techniques.

The disclosure features compositions containing at least one natural lytic protein, including isozymes, analogs, or variants thereof, isolated from the same or different bacteria, with optional additions of a complementary agent.

According to one embodiment of the disclosure, the pharmaceutical composition includes one or more altered lytic protein(s), including isozymes, analogs, or variants thereof, produced by chemical synthesis or DNA recombinant techniques. In particular, altered, lytic protein is produced by chimerization, shuffling, or both. Preferably, the pharmaceutical composition contains combination(s) of one or more natural lytic protein and one or more chimeric or shuffled lytic protein(s).

According to another embodiment of the disclosure, the pharmaceutical composition contains a peptide or a peptide fragment of at least one lytic protein derived from the same or different bacterial species, with an optional addition of one or more complementary agents, and a pharmaceutically acceptable carrier.

Also within the scope of the disclosure are compositions containing nucleic acid molecules that either alone or in combination with other nucleic acid molecules are capable of expressing an effective amount of lytic and/or holin proteins or a peptide fragment of the lytic and/or holin proteins in vivo. Also within the scope of this disclosure are cell cultures containing these nucleic acid molecules polynucleotides and vectors carrying and expressing these molecules in vitro or in vivo.

According to another embodiment of the disclosure, the pharmaceutical composition contains a complementary agent, including one or more conventional antibiotics.

According to another aspect of the disclosure, the pharmaceutical composition contains antibodies directed against a phage protein or peptide fragment of the disclosure.

The bacteriophage associated proteins of this disclosure may be administered to subjects via several means of application. Means of application include suitable carriers that assist in delivery of the composition to the site of the infection and subsequent adsorption of the composition. The compositions containing lytic and/or holin proteins or peptides and peptide fragments thereof are incorporated into pharmaceutically acceptable carriers and are placed into appropriate means of application and delivery. Preferable application means include liquid means (for example, syrups, mouthwash, and eye drops in aqueous or nonaqueous form), solid means (for example, food stuff, confectionary, and toothpaste), bandages, tampons, topical creams, among others.

In yet another embodiment of the disclosure, specific lytic proteins are used in the prevention and/or treatment of bacterial infections associated with topical or dermatological infections, administered in the form of a topical ointment or cream.

The disclosure also provides a compositiona nd method to treat or prevent infections caused by burns or wounds by using one or more phage lytic proteins, including, preferably, phage lytic proteins reactive against the various causative pathogens, and incorporating those proteins into bandages to prevent or treat infections of burns and wounds.

Embodiments of the disclosure also feature nucleic acid molecules as phage peptides and peptide fragments thereof. The nucleic acid molecules of the disclosure are preferably attached to regulatory sequences and signal sequences, wherein the sequences affect site specificity and trans-membrane movements of the nucleic acid molecules. The signal sequences affect transportation of the nucleic acid molecules to the mucous membranes.

According to another aspect of the disclosure, a method for detecting the presence of a phage protein or peptides and peptide fragments thereof of the disclosure in a sample comprises: contacting the sample with a compound which selectively binds to the phage protein or peptides and peptide fragments thereof and determining whether the compound binds to the phage protein or peptides and peptide fragments thereof in said sample. In a preferred embodiment the compound is an antibody.

In yet another embodiment of the disclosure, holin proteins are used in conjunction with phage associated lytic enzymes to prophylactically and therapeutically treat bacterial diseases. In another embodiment of the disclosure, holin proteins alone are used to prophylactically and therapeutically treat bacterial infections. The holin proteins may be shuffled holin proteins or chimeric holin proteins, in either combination with or independent of the lytic enzymes.

In yet another embodiment of the disclosure, a chimeric and/or shuffled lytic enzyme is administered through the teat into the utter of the animal.

In yet another embodiment of the disclosure, a chimeric and/or shuffled lytic enzyme is administered parenterally, wherein the phage associated lytic enzyme is administered intramuscularly, intrathecally, subdermally, subcutaneously, or intravenously to treat infections.

In yet another embodiment of the disclosure, an unaltered lytic enzyme, and/or chimeric and/or shuffled lytic enzyme is administered through the teat into the utter of the animal. The lytic enzyme in its carrier, may be injected up through the opening of the teat.

It is another object of the disclosure to apply a phage associated shuffled and/or chimeric lytic enzyme intravenously, to treat septicemia and general infections.

In yet another embodiment, chimeric lytic enzymes, shuffled lytic enzymes, “unaltered” versions of the lytic enzymes, holin proteins, and combinations thereof are used to prophylactically and therapeutically treat exposure to bacteria. In another embodiment, chimeric lytic enzymes, shuffled lytic enzymes, “unaltered” versions of the lytic enzymes, holin proteins, and combinations thereof are used to detect and identify specific bacteria. In one embodiment, the phage associated lytic enzyme specific for specific bacteria may be used to identify specific bacteria in its vegetative state.

Some sequences that have been isolated from the phage are shown in this disclosure, however other lytic enzymes from other bacteriophage specific for the same bacteria may be used in place of the sequenced lytic enzyme. In one embodiment, the DNA encoding the lytic enzyme or holin protein, including their isozymes, analogs, or variants, has been genetically altered. In another embodiment, the lytic enzyme or holin protein, including their isozymes, analogs, or variants, has been chemically altered. In yet another embodiment, the lytic enzyme or holin protein, including their isozymes, analogs, or variants, are used in a combination of natural and modified (genetically or chemically altered) forms. The altered forms of lytic enzymes and holin proteins are made synthetically by chemical synthesis and/or DNA recombinant techniques. The enzymes are made synthetically by chimerization and/or shuffling.

It should be understood that bacteriophage lytic enzymes specifically cleave bonds that are present in the peptidoglycan of bacterial cells. Since the bacterial cell wall peptidoglycan is highly conserved among all bacteria, there are only a few bonds to be cleaved to disrupt the cell wall. Enzymes that cleave these bonds are muramidases, glucosaminidases, endopeptidases, or N-acetyl-muramoyl-L-alanine amidases (hereinafter referred to as amidases). The majority of reported phage enzymes are either muramidases or amidases, and there have been no reports of bacteriophage glucosaminidases. Fischetti et al (1974) reported that the C1 streptococcal phage lysin enzyme was an amidase. Garcia et al (1987, 1990) reported that the Cp1 lysin from a S. pneumoniae from a Cp-1 phage was a lysozyme. Caldentey and Bamford (1992) reported that a lytic enzyme from the phi 6 Pseudomonas phage was an endopeptidase, splitting the peptide bridge formed by melo-diaminopimilic acid and D-alanine. The E. coli T1 and T6 phage lytic enzymes are amidases as is the lytic enzyme from Listeria phage (ply) (Loessner et al, 1996). There are also other enzymes which cleave the cell wall.

Another embodiment of the present disclosure also provides for chimeric proteins or peptides fragments which include fusion proteins for the aforesaid uses.

A definition of terms used and their applicability to the disclosure are provided as follows:

Phage enzymes or proteins, as disclosed herein, include phage polypeptides, peptide fragments, nucleic acid molecules encoding phage protein or protein peptide fragments, antibody and antibody fragments, having biological activity either alone or with combination of other molecules. When reference is made to lytic enzymes, the enzyme may include any form of the peptide that allows for the destruction of the cell wall under the specified conditions.

Nucleic acid molecules, as disclosed herein, include genes, gene fragments polynucleotides, oligonucleotides, DNA, RNA, DNA-RNA hybrids, EST, SNIPs, genomic DNA, cDNA, mRNA, antisense RNA, ribozyme vectors containing nucleic acid molecules, regulatory sequences, and signal sequences. Nucleic acid molecules of this disclosure include any nucleic acid-based molecule that either alone or in combination with other molecules produces an oligonucleotide molecule capable or incapable of translation into a peptide.

In this context of the embodiments, the term “lytic enzyme genetically coded for by a bacteriophage” means a polypeptide having at least some lytic activity against the host bacteria. The polypeptide has a sequence that encompasses a native sequence of a lytic enzyme and variants thereof. The polypeptide may be isolated from a variety of sources, such as from phage, or prepared by recombinant or synthetic methods, such as those by Garcia et al. Every polypeptide (lytic enzyme) has two domains. One domain is a cell wall binding portion at the carboxyl terminal side and the other domain is an amidase activity that acts upon amide bonds in the peptidoglycan at the amino terminal side. Generally speaking, a lytic enzyme according to the disclosure is between 25,000 and 35,000 daltons in molecular weight and comprises a single polypeptide chain; however, this can vary depending on the enzyme chain. The molecular weight is determined by assay using sodium dodecyl sulfate gel electrophoresis and comparison with molecular weight markers.

The term “isolated” means at least partially purified from a starting material. The term “purified” means that the biological material has been measurably increased in concentration by any purification process, including by not limited to, column chromatography, HPLC, precipitation, electrophoresis, etc., thereby partially, substantially or completely removing impurities such as precursors or other chemicals involved in preparing the material. Hence, material that is homogenous or substantially homogenous (e.g., yields a single protein bond in a separation procedure such as electrophoresis or chromatography) is included within the meanings of isolated and purified. The amount of purifi ation necessary will depend upon the use of the material. For example, compositions intended for administration to humans ordinarily must be highly purified in accordance with regulatory standards.

“A native sequence phage associated lytic enzyme” is a polypeptide having the same amino acid sequence as an enzyme derived from nature. This enzyme can be isolated from nature or can be produced by recombinant or synthetic means. The term “native sequence enzyme” specifically encompasses naturally occurring forms (e.g., alternatively spliced or modified forms) and naturally-occurring variants of the enzyme. In one embodiment of the disclosure, the native sequence enzyme is a mature or full-length polypeptide that is genetically coded for by a gene from a bacteriophage specific for a specific bacteria. Of course, a number of variants are possible and known, as acknowledged in publications such as Lopez et al., Microbial Drug Resistance 3: 199-211 (1997); Garcia et al., Gene 86: 81-88 (1990); Garcia et al., Proc. Natl. Acad. Sci. USA 85: 914-918 (1988); Garcia et al., Proc. Natl. Acad. Sci. USA 85: 914-918 (1988); Garcia et al., Streptococcal Genetics (J. J. Ferretti and Curtis eds., 1987); Lopez et al., FEMS Microbiol. Lett. 100: 439-448 (1992); Romero et al., J. Bacteriol. 172: 5064-5070 (1990); Ronda et al., Eur. J. Biochem. 164: 621-624 (1987) and Sanchez et al., Gene 61: 13-19 (1987). The contents of each of these references, particularly the sequence listings and associated text that compares the sequences, including statements about sequence homologies, are specifically incorporated by reference in their entireties.

“A variant sequence phage associated lytic enzyme” means a functionally active lytic enzyme genetically coded for by a bacteriophage specific for a specific bacteria, as defined below, having at least 50%, 55%, 60%, 65%, 70%, 75%, 80%, 85%, 90%, 95%, 97%, 98%, 99%, or even at least 99.5% amino acid sequence identity with the sequences shown in some of the figures. Of course a skilled artisan readily will recognize portions of this sequence that are associated with functionalities such as binding, and catalyzing a reaction. Accordingly, polypeptide sequences and nucleic acids that encode these sequences are contemplated that comprise at least 75%, 80%, 85%, 90%, 95%, 97%, 98%, 99% or more of each functional domain of some of the sequences. Such portions of the total sequence are very useful for diagnostics as well as therapeutics/prophylaxis. In fact, sequences as short as 5 amino acids long have utility as epitopic markers for the phage. More desirable, larger fragments or regions of protein having a size of at least 8, 9, 10, 12, 15 or 20 amino acids, and homologous sequences to these, have epitopic features and may be used either as small peptides or as sections of larger proteins according to embodiments. Nucleic acids corresponding to these sequences also are contemplated.

Such phage associated lytic enzyme variants include, for instance, lytic enzyme polypeptides wherein one or more amino acid residues are added, or deleted at the N or C terminus of the sequence shown. In one embodiment one or more amino acids are substituted, deleted, and/or added to any position(s) in the sequence, or sequence portion. Ordinarily, a phage associated lytic enzyme will have at least about (e.g. exactly) 50%, 55%, 60%, 65%, 70%, 75%, amino acid sequence identity with native phage associated lytic enzyme sequences, more preferably at least about (e.g. exactly) 80%, 85%, 90%, 95%, 97%, 98%, 99% or 99.5% amino acid sequence identity. In other embodiments a phage associated lytic enzyme variant will have at least about 50% (e.g. exactly 50%), 55%, 60%, 65%, 70%, 75%, 80%, 85%, 90%, 95%, 97%, 98%, 99%, or even at least 99.5% amino acid sequence identity with the sequences shown.

“Percent amino acid sequence identity” with respect to the phage associated lytic enzyme sequences identified herein is defined as the percentage of amino acid residues in a candidate sequence that are identical with the amino acid residues in the phage associated lytic enzyme sequence, after aligning the sequences in the same reading frame and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, such as using publicly available computer software such as blast software. Those skilled in the art can determine appropriate parameters for measuring alignment, including any algorithms needed to achieve maximal alignment over the whole length of the sequences being compared.

In each case, of course conservative amino acid substitutions also may be made simultaneously in determining percent amino acid sequence identity. For example, a 15 amino acid long region of protein may have 50%, 55%, 60%, 65%, 70%, 75%, 80%, 85%, 90%, 95%, 97%, 98%, or 99% sequence homology with a region of the sequences shown. At the same time, the 15 amino acid long region of the protein may also have up to 0.5%, 1%, 2%, 5%, 10%, 15%, 20%, 30%, 40%, 50%, 65%, 75%, or more amino acids replaced with conservative substitutions. Preferably the region will have fewer than 30%, 20%, 10% or even less conservative substitutions. The “percent amino acid sequence identity” calculation in such cases will be higher than the actual percent sequence identity when conservative amino acid substitutions have been made.

“Percent nucleic acid sequence identity” with respect to the phage associated lytic enzyme sequences identified herein is defined as the percentage of nucleotides in a candidate sequence that are identical with the nucleotides in the phage associated lytic enzyme sequence, after aligning the sequences and introducing gaps, if necessary, to achieve the maximum percent sequence identity. Alignment for purposes of determining percent nucleic acid sequence identity can be achieved in various ways that are within the scope of those skilled in the art, including but not limited to the use of publicly available computer software. Those skilled in the art can determine appropriate parameters for measuring alignment, including any algorithms needed to achieve maximal alignment over the full length of the sequences being compared.

“Polypeptide” refers to a molecule comprised of amino acids which correspond to those encoded by a polynucleotide sequence which is naturally occurring. The polypeptide may include conservative substitutions wherein the naturally occurring amino acid is replaced by one having similar properties, where such conservative substitutions do not alter the function of the polypeptide (see, for example, Lewin “Genes V” Oxford University Press Chapter 1, pp. 9-13 1994).

A “chimeric protein” or “fusion protein” comprises all or (preferably a biologically active) part of a polypeptide of the disclosure operably linked to a heterologous polypeptide. Chimeric proteins or peptides are produced, for example, by combining two or more proteins having two or more active sites. Chimeric protein and peptides can act independently on the same or different molecules, and hence have a potential to treat two or more different bacterial infections at the same time. Chimeric proteins and peptides also are used to treat a bacterial infection by cleaving the cell wall in more than one location.

The term “operably linked” means that the polypeptide of the disclosure and the heterologous polypeptide are fused in-frame. The heterologous polypeptide can be fused to the N-terminus or C-terminus of the polypeptide of the disclosure. Chimeric proteins are produced enzymatically by chemical synthesis, or by recombinant DNA technology. A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetration of the proteins of either the inner membrane or the inner and outer membranes of the E. coli. FEMS Microbiol. Lett. 1998 July 1, 164(1); 159-67 (incorporated herein by reference).

In another experiment, an active chimeric cell wall lytic enzyme (TSL) was constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumococcal cell walls. One example of a useful fusion protein is a GST fusion protein in which the polypeptide of the disclosure is fused to the C-terminus of a GST sequence. Such a chimeric protein can facilitate the purification of a recombinant polypeptide of the disclosure.

In another embodiment, the chimeric protein or peptide contains a heterologous signal sequence at its N-terminus. For example, the native signal sequence of a polypeptide of the disclosure can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992, incorporated herein by reference). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

Another embodiment discloses an immunoglobulin fusion protein in which all or part of a polypeptide of the disclosure is fused to sequences derived from a member of the immunoglobulin protein family. An immunoglobulin fusion protein can be incorporated into a pharmaceutical composition and administered to a subject to inhibit an interaction between a ligand (soluble or membrane-bound) and a protein on the surface of a cell (receptor), to thereby suppress signal transduction in vivo. The immunoglobulin fusion protein can alter bioavailability of a cognate ligand of a polypeptide of the disclosure. Inhibition of ligand/receptor interaction may be useful therapeutically, both for treating bacterial-associated diseases and disorders for modulating (i.e. promoting or inhibiting) cell survival. Moreover, an immunoglobulin fusion protein of the disclosure can be used as an immunogen to produce antibodies directed against a polypeptide of the disclosure in a subject, to purify ligands and in screening assays to identify molecules which inhibit the interaction of receptors with ligands. Chimeric and fusion proteins and peptides of the disclosure can be produced by standard recombinant DNA techniques.

In another embodiment, the fusion gene can be synthesized by conventional techniques, including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (see, i.e., Ausubel et al., supra). Moreover, many expression vectors are commercially available that already encode a fusion moiety (i.e., a GST polypeptide). A nucleic acid encoding a polypeptide of the disclosure can be cloned into such an expression vector such that the fusion moiety is linked in-frame to the polypeptide of the disclosure.

As used herein, shuffled proteins or peptides, gene products, or peptides for more than one related phage protein or protein peptide fragments have been randomly cleaved and reassembled into a more active or specific protein. Shuffled oligonucleotides, peptides or peptide fragment molecules are selected or screened to identify a molecule having a desired functional property. This method is described, for example, in Stemmer, U.S. Pat. No. 6,132,970. (Method of shuffling polynucleotides); Kauffman, U.S. Pat. No 5,976,862 (Evolution via Condon-based Synthesis) and Huse, U.S. Pat. No. 5,808,022 (Direct Codon Synthesis). The contents of these patents are incorporated herein by reference. Shuffling is used to create a protein that is 10 to 100 fold more active than the template protein. The template protein is selected among different varieties of lysin or holin proteins. The shuffled protein or peptides constitute, for example, one or more binding domains and one or more catalytic domains. Each binding or catalytic domain is derived from the same or a different phage or phage protein. The shuffled domains are either oligonucleotide based molecules, as gene or gene products, that either alone or in combination with other genes or gene products are translatable into a peptide fragment, or they are peptide based molecules. Gene fragments include any molecules of DNA, RNA, DNA-RNA hybrid, antisense RNA, Ribozymes, ESTs, SNIPs and other oligonucleotide-based molecules that either alone or in combination with other molecules produce an oligonucleotide molecule capable or incapable of translation into a peptide.

As noted above, the present disclosure discusses the use of holin proteins. Holin proteins produce holes in the cell membrane. More specifically, holins form lethal membrane lesions. Like the lytic proteins, holin proteins are coded for and carried by a phage. In fact, it is quite common for the genetic code of the holin protein to be next to or even within the code for the phage lytic protein. Most holin protein sequences are short, and overall, hydrophobic in nature, with a highly hydrophilic carboxy-terminal domain. In many cases, the putative holin protein is encoded on a different reading frame within the enzymatically active domain of the phage. In other cases, holin protein is encoded on the DNA next or close to the DNA coding for the cell wall lytic protein. Holin proteins are frequently synthesized during the late stage of phage infection and found in the cytoplasmic membrane where they cause membrane lesions.

Holins can be grouped into two general classes based on primary structure analysis. Class I holins are usually 95 residues or longer and may have three potential transmembrane domains. Class II holins are usually smaller, at approximately 65-95 residues, with the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all phages. Holins have been shown to be present in several bacteria, including, for example, lactococcal bacteriophage Tuc2009, lactococcal NLC3, pneumococcal bacteriophage EJ-1, Lactobacillus gasseri bacteriophage Nadh, Staphylococcus aureus bacteriophage Twort, Listeria monocytogenes bacteriophages, pneumococcal phage Cp-1, Bacillus subtillis phage M29, Lactobacillus delbrueckki bacteriophage LL-H lysin, and bacteriophage N11 of Staphyloccous aureus. (Loessner, et al., Journal of Bacteriology, August 1999, p. 4452-4460).

The modified or altered form of the protein or peptides and peptide fragments, as disclosed herein, includes protein or peptides and peptide fragments that are chemically synthesized or prepared by recombinant DNA techniques, or both. These techniques include, for example, chimerization and shuffling. When the protein or peptide is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

In one embodiment of the disclosure, a signal sequence of a polypeptide can facilitate transmembrane movement of the protein and peptides and peptide fragments of the disclosure to and from mucous membranes, as well as by facilitating secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the disclosure can pertain to the described polypeptides having a signal sequence, as well as to the signal sequence itself and to the polypeptide in the absence of the signal sequence (i.e., the cleavage products). In one embodiment, a nucleic acid sequence encoding a signal sequence of the disclosure can be operably linked in an expression vector to a protein of interest, such as a protein which is ordinarily not secreted or is otherwise difficult to isolate. The signal sequence directs secretion of the protein, such as from an eukaryotic host into which the expression vector is transformed, and the signal sequence is subsequently or concurrently cleaved. The protein can then be readily purified from the extracellular medium by art recognized methods. Alternatively, the signal sequence can be linked to a protein of interest using a sequence which facilitates purification, such as with a GST domain.

In another embodiment, a signal sequence can be used to identify regulatory sequences, i.e., promoters, enhancers, repressors. Since signal sequences are the most amino-terminal sequences of a peptide, it is expected that the nucleic acids which flank the signal sequence on its amino-terminal side will be regulatory sequences that affect transcription. Thus, a nucleotide sequence which encodes all or a portion of a signal sequence can be used as a probe to identify and isolate the signal sequence and its flanking region, and this flanking region can be studied to identify regulatory elements therein. The present disclosure also pertains to other variants of the polypeptides of the disclosure. Such variants have an altered amino acid sequence which can function as either agonists (mimetics) or as antagonists. Variants can be generated by mutagenesis, i.e., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein. Variants of a protein of the disclosure which function as either agonists (mimetics) or as antagonists can be identified by screening combinatorial libraries of mutants, i.e., truncation mutants, of the protein of the disclosure for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (i.e., for phage display). There are a variety of methods which can be used to produce libraries of potential variants of the polypeptides of the disclosure from a degenerate oligonucleotide sequence. Methods for synthesizing degenerate oligonucleotides are known in the art (see, i.e., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477, all herein incorporated by reference).

In addition, libraries of fragments of the coding sequence of a polypeptide of the disclosure can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest. Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the disclosure (Arkin and Yourvan (1992) Proc. Natl. Acad. Sci. USA 89:7811-7815; Delgrave et al. (1993) Protein Engineering 6(3):327-331).

Immunologically active portions of a protein or peptide fragment include regions that bind to antibodies that recognize the phage enzyme. In this context, the smallest portion of a protein (or nucleic acid that encodes the protein) according to embodiments is an epitope that is recognizable as specific for the phage that makes the lysin protein. Accordingly, the smallest polypeptide (and associated nucleic acid that encodes the polypeptide) that can be expected to bind antibody and is useful for some embodiments may be 8, 9, 10, 11, 12, 13, 15, 20, 25, 30, 35, 40, 45, 50, 55, 60, 65, 75, 85, or 100 amino acids long. Although small sequences as short as 8, 9, 10, 11, 12 or 15 amino acids long reliably comprise enough structure to act as epitopes, shorter sequences of 5, 6, or 7 amino acids long can exhibit epitopic structure in some conditions and have value in an embodiment. Thus, the smallest portion of the protein or nucleic acid sequence described by specific sequences includes polypeptides as small as 5, 6, 7, 8, 9, or 10 amino acids long.

Homologous proteins and nucleic acids can be prepared that share functionality with such small proteins and/or nucleic acids (or protein and/or nucleic acid regions of larger molecules) as will be appreciated by a skilled artisan. Such small molecules and short regions of larger molecules, that may be homologous specifically are intended as embodiments. Preferably the homology of such valuable regions is at least 50%, 65%, 75%, 85%, and more preferably at least 90%, 95%, 97%, 98%, or at least 99% compared to the specific sequences. These percent homology values do not include alterations due to conservative amino acid substitutions.

Of course, an epitope as described herein may be used to generate an antibody and also can be used to detect binding to molecules that recognize the lysin protein. Another embodiment is a molecule such as an antibody or other specific binder that may be created through use of an epitope such as by regular immunization or by a phase display approach where an epitope can be used to screen a library of potential binders. Such molecules recognize one or more epitopes of lysin protein or a nucleic acid that encodes lysin protein. An antibody that recognizes an epitope may be a monoclonal antibody, a humanized antibody, or a portion of an antibody protein. Desirably the molecule that recognizes an epitope has a specific binding for that epitope which is at least 10 times as strong as the molecule has for serum albumin. Specific binding can be measured as affinity (Km). More desirably the specific binding is at least 10², 10³, 10⁴, 10⁵, 10⁶, 10⁷, 10⁸, or even higher than that for serum albumin under the same conditions.

In a desirable embodiment the antibody or antibody fragment is in a form useful for detecting the presence of the lysin protein. A variety of forms and methods for their synthesis are kno The antibody may be conjugated (covalently complexed) with a reporter molecule or atom such as a fluor, an enzyme that creates an optical signal, a chemilumiphore, a microparticle, or a radioactive atom. The antibody or antibody fragment may be synthesized in vivo, after immunization of an animal, for example, The antibody or antibody fragment may be synthesized via cell culture after genetic recombination. The antibody or antibody fragment may be prepared by a combination of cell synthesis and chemical modification.

Biologically active portions of a protein or peptide fragment of the embodiments, as described herein, include polypeptides comprising amino acid sequences sufficiently identical to or derived from the amino acid sequence of the phage protein of the disclosure, which include fewer amino acids than the full length protein of the phage protein and exhibit at least one activity of the corresponding full-length protein. Typically, biologically active portions comprise a domain or motif with at least one activity of the corresponding protein. A biologically active portion of a protein or protein fragment of the disclosure can be a polypeptide which is, for example, 10, 25, 50, 100 less or more amino acids in length. Moreover, other biologically active portions, in which other regions of the protein are deleted, or added can be prepared by recombinant techniques and evaluated for one or more of the functional activities of the native form of a polypeptide of the embodiments.

A large variety of isolated cDNA sequences that encode phage associated lysing enzymes and partial sequences that hybridize with such gene sequences are useful for recombinant production of the lysing enzyme. Representative nucleic acid sequences in this context are the sequences shown in the figures and sequences that hybridize, under stringent conditions, with complementary sequences of the DNA from those sequences. Still further variants of these sequences and sequences of nucleic acids that hybridize with those shown in the figures also are contemplated for use in production of lysing enzymes according to the disclosure, including natural variants that may be obtained.

Many of the contemplated variant DNA molecules include those created by standard DNA mutagenesis techniques, such as M13 primer mutagenesis. Details of these techniques are provided in Sambrook et al. (1989) In Molecular Cloning: A Laboratory Manual, Cold Spring Harbor, N.Y. (incorporated herein by reference). By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristic of the BSMR protein are contemplated by the disclosure. Also included are one small DNA molecules which are derived from the disclosed DNA molecules. Such small DNA molecules include oligonucleotides suitable for use as hybridization probes or polymerase chain reaction (PCR) primers. As such, these small DNA molecules will comprise at least a segment of a lytic enzyme genetically coded for by a bacteriophage specific for a specific bacteria and, for the purposes of PCR, will comprise at least a 10-15 nucleotide sequence and, more preferably, a 15-30 nucleotide sequence of the gene. DNA molecules and nucleotide sequences which are derived from the disclosed DNA molecules as described above may also be defined as DNA sequences which hybridize under stringent conditions to the DNA sequences disclosed, or fragments thereof.

Hybridization conditions corresponding to particular degrees of stringency vary depending upon the nature of the hybridization method of choice and the composition and length of the hybridizing DNA used. Generally, the temperature of hybridization and the ionic strength (especially the sodium ion concentration) of the hybridization buffer will determine the stringency of hybridization. Calculations regarding hybridization conditions required for attaining particular degrees of stringency are discussed by Sambrook et al. (1989), In Molecular Cloning: A Laboratory Manual, Cold Spring Harbor, N.Y., chapters 9 and 11, (herein incorporated by reference).

An example of such calculation is as follows. A hybridization experiment may be performed by hybridization of a DNA molecule (for example, a natural variation of the lytic enzyme genetically coded for by a bacteriophage specific for Bacillus anthracis) to a target DNA molecule. A target DNA may be, for example, the corresponding cDNA which has been electrophoresed in an agarose gel and transferred to a nitrocellulose membrane by Southern blotting (Southern (1975). J. Mol. Biol. 98:503), a technique well known in the art and described in Sambrook et al. (1989) In Molecular Cloning: A Laboratory Manual, Cold Spring Harbor, N.Y. (incorporated herein by reference). Hybridization with a target probe labeled with isotopic P (32) labeled-dCTP is carried out in a solution of high ionic strength such as 6 times SSC at a temperature that is 20-25 degrees Celsius below the melting temperature, Tm, (described infra). For such Southern hybridization experiments where the target DNA molecule on the Southern blot contains 10 ng of DNA or more, hybridization is carried out for 6-8 hours using 1-2 ng/ml radiolabeled probe (of specific activity equal to 109 CPM/mug or greater). Following hybridization, the nitrocellulose filter is washed to remove background hybridization. The washing conditions are as stringent as possible to remove background hybridization while retaining a specific hybridization signal. The term “Tm” represents the temperature above which, under the prevailing ionic conditions, the radiolabeled probe molecule will not hybridize to its target DNA molecule.

The Tm of such a hybrid molecule may be estimated from the following equation: Tm=81.5 degrees C.−16.6(log10 of sodium ion concentration)+0.41(% G+C)−0.63(% formamide)−(600/1) where l=the length of the hybrid in base pairs. This equation is valid for concentrations of sodium ion in the range of 0.01M to 0.4M, and it is less accurate for calculations of Tm in solutions of higher sodium ion concentration (Bolton and McCarthy (1962). Proc. Natl. Acad. Sci. USA 48:1390) (incorporated herein by reference). The equation also is valid for DNA having G+C contents within 30% to 75%, and also applies to hybrids greater than 100 nucleotides in length. The behavior of oligonucleotide probes is described in detail in Ch. 11 of Sambrook et al. (1989), In Molecular Cloning: A Laboratory Manual, Cold Spring Harbor, N.Y. (incorporated herein by reference). The preferred exemplified conditions described here are particularly contemplated for use in selecting variations of the lytic gene.

Thus, by way of example, of a 150 base pair DNA probe derived from the first 150 base pairs of the open reading frame of a cDNA having a % GC=45%, a calculation of hybridization conditions required to give particular stringencies may be made as follows:

Assuming that the filter will be washed in 0.3 X SSC solution following hybridization, sodium ion=0.045M; % GC=45%; Formamide concentration=01=150 base pairs (see equation in Sambrook et al.) and so Tm=74.4 degrees C. The Tm of double-stranded DNA decreases by 1-1.5 degrees C. with every 1% decrease in homology (Bonner et al. (1973). J. Mol. Biol. 81:123). Therefore, for this given example, washing the filter in 0.3 times SSC at 59.4-64.4 degrees C. will produce a stringency of hybridization equivalent to 90%; DNA molecules with more than 10% sequence variation relative to the target BSMR cDNA will not hybridize. Alternatively, washing the hybridized filter in 0.3 times SSC at a temperature of 65.4-68.4 degrees C. will yield a hybridization stringency of 94%; DNA molecules with more than 6% sequence variation relative to the target BSMR cDNA molecule will not hybridize. The above example is given entirely by way of theoretical illustration. One skilled in the art will appreciate that other hybridization techniques may be utilized and that variations in experimental conditions will necessitate alternative calculations for stringency.

In preferred embodiments of the present disclosure, stringent conditions may be defined as those under which DNA molecules with more than 25% sequence variation (also termed “mismatch”) will not hybridize. In a more preferred embodiment, stringent conditions are those under which DNA molecules with more than 15% mismatch will not hybridize, and more preferably still, stringent conditions are those under which DNA sequences with more than 10% mismatch will not hybridize. Preferably, stringent conditions are those under which DNA sequences with more than 6% mismatch will not hybridize.

The degeneracy of the genetic code further widens the scope of the embodiments as it enables major variations in the nucleotide sequence of a DNA molecule while maintaining the amino acid sequence of the encoded protein. For example, a representative amino acid residue is alanine. This may be encoded in the cDNA by the nucleotide codon triplet GCT. Because of the degeneracy of the genetic code, three other nucleotide codon triplets—GCT, GCC and GCA—also code for alanine. Thus, the nucleotide sequence of the gene could be changed at this position to any of these three codons without affecting the amino acid composition of the encoded protein or the characteristics of the protein. The genetic code and variations in nucleotide codons for particular amino acids are well known to the skilled artisan. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein comprehended by this disclosure.

One skilled in the art will recognize that the DNA mutagenesis techniques described here can produce a wide variety of DNA molecules that code for a bacteriophage lysin specific for a specific bacteria yet that maintain the essential characteristics of the lytic protein. Newly derived proteins may also be selected in order to obtain variations on the characteristic of the lytic protein, as will be more fully described below. Such derivatives include those with variations in amino acid sequence including minor deletions, additions and substitutions.

While the site for introducing an amino acid sequence variation is predetermined, the mutation per se does not need to be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically of single residues; insertions usually will be on the order of about from 1 to 10 amino acid residues; and deletions will range about from 1 to 30 residues. Deletions or insertions may be in single form, but preferably are made in adjacent pairs, i.e., a deletion of 2 residues or insertion of 2 residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary mRNA structure (EP 75,444A). Substitutional variants are those in which at least one residue in the amino acid sequence has been removed and a different residue inserted in its place. Such substitutions may be made in accordance with the following Table 1 when it is desired to finely modulate the characteristics of the protein. Table 1 shows amino acids which may be substituted for an original amino acid in a protein and which are regarded as conservative substitutions.

TABLE 1

| Original Residue | Conservative Substitutions |
|---|---|
| Ala | ser |
| Arg | lys |
| Asn | gln, his |
| Asp | glu |
| Cys | ser |
| Gln | asn |
| Glu | asp |
| Gly | pro |
| His | asn; gln |
| Ile | leu, val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than in Table 1, i.e., selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions which in general are expected to produce the greatest changes in protein properties will be those in which: (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The effects of these amino acid substitutions or deletions or additions may be assessed for derivatives of the lytic protein by analyzing the ability of the derivative proteins to complement the sensitivity to DNA cross-linking agents exhibited by phages in infected bacteria hosts. These assays may be performed by transfecting DNA molecules encoding the derivative proteins into the bacteria as described above.

Having herein provided nucleotide sequences that code for lytic enzyme genetically coded for by a bacteriophage specific for a specific bacteria and fragments of that enzyme, correspondingly provided are the complementary DNA strands of the cDNA molecule and DNA molecules which hybridize under stringent conditions to the lytic enzyme cDNA molecule or its complementary strand. Such hybridizing molecules include DNA molecules differing only by minor sequence changes, including nucleotide substitutions, deletions and additions. Also contemplated by this disclosure are isolated oligonucleotides comprising at least a segment of the cDNA molecule or its complementary strand, such as oligonucleotides which may be employed as effective DNA hybridization probes or primers useful in the polymerase chain reaction. Hybridizing DNA molecules and variants on the lytic enzyme cDNA may readily be created by standard molecular biology techniques.

The detection of specific DNA mutations may be achieved by methods such as hybridization using specific oligonucleotides (Wallace et al. (1986). Cold Spring Harbor Symp. Quant. Biol. 51:257-261), direct DNA sequencing (Church and Gilbert (1988). Proc. Natl. Acad. Sci. USA 81:1991-1995), the use of restriction enzymes (Flavell et al. (1978). Cell 15:25), discrimination on the basis of electrophoretic mobility in gels with denaturing reagent (Myers and Maniatis (1986). Cold Spring Harbor Symp. Quant. Biol. 51:275-284), RNase protection (Myers et al. (1985). Science 230:1242), chemical cleavage (Cotton et al. (1985). Proc. Natl. Acad. Sci. USA 85:4397-4401) (incorporated herein by reference), and the ligase-mediated detection procedure (Landegren et al., 1988).

Oligonucleotides specific to normal or mutant sequences are chemically synthesized using commercially available machines, labeled radioactively with isotopes (such as .sup.32 P) or non-radioactively (with tags such as biotin (Ward and Langer et al. Proc. Natl. Acad. Sci. USA 78:6633-6657 1981) (incorporated herein by reference), and hybridized to individual DNA samples immobilized on membranes or other solid supports by dot-blot or transfer from gels after electrophoresis. The presence or absence of these specific sequences are visualized by methods such as autoradiography or fluorometric or calorimetric reactions (Gebeyehu et al. Nucleic Acids Res. 15:4513-4534 1987) (incorporated herein by reference).

Sequence differences between normal and mutant forms of the gene may also be revealed by the direct DNA sequencing method of Church and Gilbert (1988) (incorporated herein by reference). Cloned DNA segments may be used as probes to detect specific DNA segments. The sensitivity of this method is greatly enhanced when combined with PCR (Stoflet et al. Science 239:491-494, 1988) (incorporated herein by reference). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags. Such sequences are useful for production of lytic enzymes according to embodiments of the disclosure.

Additional objects and advantages embodiments found in the disclosure will be set forth in the description which follows, and in part will be obvious from the description, or may be learned by practice of the embodiments. The objects and advantages of the disclosure may be realized and obtained by means of the instrumentalities and combinations particularly pointed out in the appended claims.

### BRIEF DESCRIPTION OF THE DRAWINGS

FIG. 1 is an electron micrograph of group A streptococci treated with lysin showing the collapse of the cell wall and the cell contents pouring out.

### DETAILED DESCRIPTION OF THE DISCLOSURE

Prophylactic and therapeutic compositions are disclosed that contain as an active ingredient one or more bacteria-associated phage proteins or protein peptides fragments, including isozymes, analogs, or variants of phage enzymes or phage peptides and peptide fragments thereof in a natural or modified form as active drugs and the method of use for such compositions for the treatment of mastitis. The bacteria-associated phage proteins, include a variety of bacteria-specific phage lytic and holin proteins that are derived from one or several bacterial species.

Bacteriophage lytic proteins specifically cleave bonds that are present in the peptidoglycan of bacterial cells. Since the peptidoglycan is highly conserved among all bacteria, there are only a few bonds to be cleaved to disrupt the cell wall. Proteins having the ability to hydrolyze components of a bacterial peptidoglycan fall into one of four categories:

1. N-acetylmuramoyl-L-alanine amidases (E.C. 3.5.1.28)—These proteins hydrolyze the link between N-acetylmuramoyl residues and L-amino acid residues in certain bacterial cell-wall glycopeptides.

Streptococcal lysin belongs to this family of lytic proteins. Of the 27 sequenced amidases, only the five highlighted are of bacteriophage origin. The rest are autolysins of bacterial origin.

2. Lysozyme. (EC 3.2.1.17), also known as muramidase. This protein hydrolyses the 1,4-beta-linkages between N-acetyl-D-glucosamine and N-acetylmuramic acid in peptidoglycan heteropolymers of the prokaryotes cell walls.

Of the 94 known sequences, 15 are encoded by bacteriophages.

3. Beta 1,4 N-acetyl-D-glucosaminidase (EC 3.2.1.14), also known as chitinase or chitodextrinase. Hydrolysis of the 1_{—}4-beta-linkages of -acetyl-D-glucosamine polymers of chitin. These proteins are found primarily in the plant kingdom, although some are found in bacteria. None of the 104 known proteins are encoded by bacteriophages. However, many of these proteins that are produced by bacteria also possess lysozyme activity, and are usually classified with the other lysozymes.

4. Endopeptidase that cleaves the cross bridge of the peptidoglycan. The only known endopeptidase to be characterized extensively which acts on the peptidoglycan is lysostaphin (EC 3.4.24.75). This is a metalloprotease that hydrolyses the -Gly-l-Glybond in the pentaglycine inter-peptide link joining staphylococcal cell wall peptidoglycans. This protein is found in several streptococcal species, but it is not encoded by bacteriophages. The only reported phage encoded endopeptidase that acts on the peptidoglycan is from a Pseudomonas phi 6 phage.

The majority of reported phage proteins are either muramidases or amidases. Fischetti et al (1974) reported that the C1 streptococcal phage lysine protein was an amidase. Garcia et al (1987, 1990) reported that the CP-1 lysin from an S. pneumoniae phage was a muramidase. Caldentey and Bamford (1992) reported that a lytic protein from the phi 6 Pseudomonas phage was an endopeptidase, splitting the peptide bridge formed by meso-diaminopimilic acid and D-alanine. The E. coli T1 and T6 phage lytic proteins are amidases as is the lytic protein from Listeria phage (ply) (Loessner et al 1996).

Bacteriophages

There are a large number of phages which will attach to specific bacteria and produce enzymes which will lyse that particular bacteria. The following are a list of bacteriophages and bacteria for which they are specific and which can be treated when infecting a body:

Actinomycetes

Actinomyces israelii

Agrobacterium

Alcaligenes

Bacillus

Bacillus anthracis

Bacteroides

Bacteroides fragilis

Bartonella

Bartonella bacilliformis

Bartonella henselae

Bdellovibrio

Bordetella

Bordetella pertussis

Borrelia

Borrelia burdorferi

Borrelia recurrentis

Brucella

Brucella abortus

Brucella melitensis

Brucella suis

Burkholderia

Calymmatobacterium

Calymmatobacterium donovani

Campylobacter

Campylobacter fetus

Campylobacter jejuni

Caulobacter

Clostridium

Clostridium botulinum

Clostridium difficile

Clostridium perfringens

Clostridium septicum

Clostridium tetani

Corynebacteria

Corynebacterium diptheriae

Coryneforms

Cyanobacteria

Enterobacteria

Enterobacter (Aerobacter) aerogenes

Escherschia coli

Francisella

Francisella tularensis

Haemophilus

Haemophilus ducreyi

Haemophilus influenzae

Klebsiella

Klebisiella ozaenae

Klebsiella pneumoniae

Klebsiella rhinoscleromatis

Lactobacillus

Lactoctococcus

Legionella

Legionella pneumophila

Leptospira

Listeria

Listeria monocytogenes

Micrococcus

Mollicutes

Mycobacteria

Mycobacterium avium

Mycobacterium bovis

Mycobacterium intracellulare

Mycobacterium kansasii

Mycobacterium leprae

Mycobacterium tuberculosis

Mycobacterium ulcerans

Myxococcus

Neisseria

Neisseria gonorrhoeae

Neisseria meningitidis

Pasteurella

Pneumococci

Proteus

Proteus mirabilis

Proteus morgagni

Pseudomonas

Pseudomonas aeruginosa

Pseudomonas mallei

Pseudomonas pseudomalli

Rhizobium

Salmonella

Salmonella typhi

Salmonella typhimurium

Serratia

Serratia marcescens

Shigella

Spirillum

Spirillum minus

Spirochete

Spiroplasma

Staphylococci

Staphylococcus aureus

Streptobacillus

Streptobacillus moniliformis

Streptococci

Streptococcus pyogenes

Streptococcus pneumoniae

Treponema

Treponema carateum

Treponema pallidum

Treponema pertenue

Vibrio

Vibrio cholerae

Xanthomonas

Yersinia

Yersinia enterocolitica

Yersinia pestis

Various phages which can be used to infect these bacteria and create the lytic enzyme include:

Bacteriaphage(s)

Acholeplasma BN1, MV-L3, (syn=MVL3), MVL51, MVL52, MV-L59, MV-L60, 03cl, 011clr, 10tur, 143tur, 179tur, 182tur, 1304clr, MV-L1
AchromobacterOXN-36P, NN-Achromobacter (1)
AcinetobacterA31, A33, A34, A36, A37, BP1, B₉GP, P78, 56. 142, 205, E4, E5, HP1, 102, 106, 133, A1, A3/2, A9, A10/45, BS46, E1, E2, E7, E14, G4, HP2, HP3, HP4, 20, 59, 73, 103, 104, 108, 138, 141, 143, 196, 204, 206, E6, E8, E9, E13, E15, 1, 11, 66
ActinobacillusAaφ23, Aaφ76, Aaφ97, Aaφ99, Aaφ247, PAA24, PAA84, φAa17, NN-Actinobacillus(1) PAA17, PAA23, NN-Actinobacillus (2)
ActinomycetesAv-1, Av-2, Av-3, BF307, CT1, CT2, CT3, CT4, CT8, CT6, CT7, 1281
Aeromonas AA-1, 29, 37, 43, 51, 59.1, Aeh1, F, PM2, 1, 25, 31, 40RR2.8t, (syn=44R), (syn=44RR_{2.8}t), 65, Aeh2, N, PM1, TP446, 3, 4, 11, 13, 29, 31, 32, 37, 43, 43-10T, 51, 54, 55R. 1, 56, 56RR2, 57, 58, 59.1, 60, 63, PM3, PM4, PM5, PM6
Altermonas PM2
Bacillus 4 (B. megaterium), 4 (B. sphaericus) ale1, AR1, AR2, AR3, AR7, AR9, Bace-11, (syn=11), Bastille, BL1, BL2, BL3, BL4, BL5, BL6, BL8, BL9, BP124, BS28, BS80, Ch, CP-51, CP-54, D-5, dar1, den1, DP-7, ent2, FoS₁, FoS₂, FS₄, FS₆, FS₇, G, gal1, gamma, GE1, GF-2, GS₁, GT-1, GT-2, GT-3, GT-4, GT-5, GT-6, GT-7, GV-6, g15, I9, I10, IS₁, K, MP9, MP13, MP21, MP23, MP24, MP28, MP29, MP30, MP32, MP34, MP36, MP37, MP39, MP40, MP41, MP43, MP44, MP45, MP47, MP50, NLP-1, No. 1, N17, N19, PBS1, PK1, PMB1, PMB12, PMJ1, S, SPO1, SP3, SP5, SP6, SP7, SP8, SP9, SP10, SP-15, SP50, (syn=SP-50), SP82, SST, sub1, SW, Tg8, Tg12, Tg13, Tg14, thu1, thu4, thu5, Tin4, Tin23, TP-13, TP33, TP50, TSP-1, type V, type VI, V, Vx, β22, φe, φNR2, φ25, φ63, 1, 1, 2, 2C, 3NT, 4, 5, 6, 7, 8, 9, 10, 12, 12, 17, 18, 19, 21, 138, III, AR13, BPP-10, BS32, BS107, B1, B2, GA-1, GP-10, GV-3, GV-5, g8, MP20, MP27, MP49, Nf, PP5, PP6, SF5, Tg18, TP-1, Versailles, φ15, φ29, 1-97, 837/IV, NN-Bacillus (1) A, aiz1, Al—K—I, B, BCJA1, BC1, BC2, BLL1, BL1, BP142, BSL1, BSL2, BS1, BS3, BS8, BS15, BS18, BS22, BS26, BS28, BS31, BS104, BS105, BS106, BTB, B1715V1, C, CK-1, Col1, Cor1, CP-53, CS-1, CS₁, D, D, D, D5, ent1, FP8, FP9, FS₁, FS₂, FS₃, FS₅, FS₈, FS₉, G, GH8, GT8, GV-1, GV-2, GT-4, g3, g12, g13, g14, g16, g17, g21, g23, g24, g29, H2, ken1, KK-88, Kum1, Kyu1, J7W-1, LP52, (syn=LP-52), L₇, Mex1, MJ-1, mor2, MP-7, MP10, MP12, MP14, MP15, Neo1, N°2, N5, N6P, PBC1, PBLA, PBP1, P2, S-a, SF2, SF6, Sha1, Sil1, SPO2, (syn=ΦSPP1), SPβ, STI, ST₁, SU-11, t, Tb1, Tb2, Tb5, Tb10, Tb26, Tb51, Tb53, Tb55, Tb77, Tb97, Tb99, Tb560, Tb595, Td8, Td6, Td15, Tg1, Tg4, Tg6, Tg7, Tg9, Tg10, Tg11, Tg13, Tg15, Tg2l, Tin1, Tin7, Tin8, Tin13, Tm3, Toc1, Tog1, tol1, TP-1, TP-10ᵥᵢᵣ, TP-15c, TP-16c, TP-17c, TP-19, TP35, TP51, TP-84, Tt4, Tt6, type A, type B, type C, type D, type E, Tφ3, VA-9, W, wx23, wx26, Yun1, α, γ, ρ11, φmed-2, φT, φμ-4, φ3T, φ75, φ105, (syn=φ105), 1A, 1B, 1-97A, 1-97B, 2, 2, 3, 3, 3, 5, 12, 14, 20, 30, 35, 36, 37, 38, 41C, 51, 63, 64, 138D, I, II, IV, NN-Bacillus (13), Bat10, BSL10, BSL11, BS6, BS11, BS16, BS23, BS101, BS102, g18, mor1, PBL1, SN45, thu2, thu3, Tm1, Tm2, TP-20, TP21, TP52, type F, type G, type IV, NN-Bacillus (3) BLE, (syn=θc), BS2, BS4, BS5, BS7, B10, B12, BS20, BS21, F, MJ-4, PBA12, AP50, AP50-04, AP50-11, AP50-23, AP50-26, AP50-27, Bam35
BacteroidesBf-41, ad1₂, Baf-44, Baf-48B, Baf-64, Bf-1, Bf-52, B40-8, F1, β1, φA1, φBr01, φBr02, 67.1, 67.3, 68.1, NN-Bacteroides (3)
BdellovibrioMAC-1, MAC-1′, MAC-2, MAC-4, MAC-4′, MAC-5, MAC-7, MAC-1, MAC-1′, MAC-2, MAC-4, HDC-2, MAC-6, VL-1
BacteroidesBf42, Bf71, NN-Bdellovibrio (1)
BorreliaNN-Borrelia (2), NN-Borrelia (1)
BrucellaA422, Bk, (syn=Berkeley), BM₂₉, FO₁, (syn=FO₁), (syn=FQ1), D, FP₂, (syn=FP2), (syn=FD2), Fz, (syn=Fz75/13), (syn=Firenze 75/13), (syn=Fi), F₁, (syn=F1), F₁m, (syn=F1m), (syn=Fim), F₁U, (syn=F1U), (syn=FiU), F₂, (syn=F2), F₃, (syn=F3), F₄, (syn=F4), F₅, (syn=F5), F₆, F₇, (syn=F7), F₂₅, (syn=F25), (syn=f25), F₂₅U, (syn=F₂₅u), (syn=F25U), (syn=F25V), F₄₄, (syn=F44), F₄₅, (syn=F45), F₄₈, (syn=F48), I, Im, M, MC/75, M51, (syn=M85), P, (syn=D), S708, R, Tb, (syn=TB), syn=Tbilisi), W, (syn=Wb), (syn=Weybridge), X, 3, 6, 7, 10/1, (syn=10), (syn=F₈), (syn=F8), 12m, 24/11, (syn=24), (syn=F₉), (syn=F9), 45/III, (syn=45), 75, 84, 212/XV, (syn=212), (syn=F₁₀), (syn=F10), 371/XXIX, (syn=371), syn=F₁₁), (syn=F11), 513
BurkholderiaCP75, NN-Burkholderia (1)
CampylobacterC type, NTCC12669, NTCC12670NTCC12671, NTCC12672, NTCC12673NTCC12674, NTCC12675, NTCC12676NTCC12677, NTCC12678, NTCC12679NTCC12680, NTCC12681, NTCC12682, NTCC12683, NTCC12684, 32f, 111c, 191NN-Campylobacter (2), Vfi-6, (syn=V19), Vfv-3V2, V3, V8, V16, (syn=Vfi-1), V19, V20(V45), V45, (syn=V-45) NN-Campylobacter (1)
CaulobacterφCr24, φCr26, φCr30, φCr35, φCb5, φCb8r φCb12r, φCb23r, φCp2, φCp14, φCr14, φCr28φCd1, φCr40, φCr41, φCr1, φCr22, φ101, φ102φ118, φ151, φ6, 76, φCbK, φCb3, φCb6φCb13, φCp34, φCr2, φCr4, φCr5, φCr6, φCr7,_φCr8, φCr9, φCr10, φCr11, φCr12, φCr13, φCr15, φCr20, φCr21, φCr23, φCr25, φCr27, φCr29, φCr31, φCr32, φCr33, φCr34, φCr36,_φCr37, φCr38, φCr39, φCr42, φCr43
Citrobacter FC3-9, FC3-8
Clostridium F1, HM7, HM3, CEB, CA5, Ca7, CEβ, (syn=1C), CEγ, Cld1, c-n71, c-203 Tox−, DEβ(syn=1D), (syn=1D^{tox+}), HM3, KM1, KT, Ms, NA1, (syn=Na1^{tox+}), PA1350e, Pfö, PL73, PL78, PL81, P1, P50, P5771, P19402, 1C^{tox+}, 2C^{tox-}, 2D, (syn=2D^{tox+}), 3C, (syn=3C^{tox+}), 4C, (syn=4C^{tox+}), 56, III-1, NN-Clostridium (61) CAK1, CA1, HMT, HM2, PF1, P-₂₃, P-₄₆, Q-₀₅ Q-₀₆, Q-₁₆, Q-₂₁, Q-₂₆, Q-₄₀, Q-₄₆, S₁₁₁, SA₀₂WA₀₁, WA₀₃, W₁₁₁, W₅₂₃, 80, C, CA2, CA3, CPT1, CPT4, c1, c4, c5, HM7, H₁₁/A₁, H₁₈/A₁H₂₂/S₂₃, H₁₅₈/A₁, K₂/A₁, Kub21/S₂₃, M_{L}, NA2^{tox-}, Pf2, Pf3, Pf4, S₉/S₃, S₄₁/A₁, S₄₄/S₂₃, α2, 41, 112/S₂₃, 214/S₂₃, 233/A₁, 234/S₂₃, 235/S₂₃, II-1, II-2, II-3 CA1, F1, K, S2, 1, 5, NN-Clostridium (8) NN-Clostridium (12)
ColiformAE2, dA, Ec9, f1, fd, HR, M13, ZG/2, ZJ/2
CoryneformsArp, BL3, CONX, MT, Beta, A8010, A19, A A2, A3, A101, A128, A133, A137, A139, A155, A182, B, BF, B17, B18, B51, B271, B275, B276, B277, B279, B282, C, cap₁, CC1, CG1, CG2, CG33, CL31, Cog, (syn=CG5), D, E, F, H, H-1, hq₁, hq₂, I₁/H₃₃, I₁/31, J, K, K, (syn=K^{tox-}), L, L, (syn=L^{tox+}), M, MC-1, MC-2, MC-3, MC-4, MLMa, N, O, ov₁, ov₂, ov₃, P, P, R, RP6, R_{S}29, S, T, U, UB₁, ub₂, UH₁, UH₃, uh₃, uh₅, uh₆, β, (syn=β^{tox+}), βₕᵥ₆₄, βvir, γ, (syn=γ^{tox-}), γ19, δ, (syn=δ^{tox+}), ρ, (syn=ρ^{tox-}), φ9, φ984, ω, 1A, 1/1180, 2, 2/1180, 5/1180, 5ad/9717, 7/4465, 8/4465, 8ad/10269, 0/9253, 13/9253, 15/3148, 21/9253, 28, 29, 55, 2747, 2893, 4498, 5848. CGK1
Cyanobacteria S-2L, S-4L, N1, AS-1, S-6(L)
EnterobacterC3, WS-EO20, WS-EP26, WS-EP28, φmp 667/617, 886 C-2, If1, f2, Ike, I2-2, PR64FS, SF, tf-1, PRD1, H-19J, B6, B7, C-1, C2, Jersey, ZG/3A, T5, ViII, WS-EP57, 379/319 b4, chi, Beccles, tu, PRR1, 7s, C-1, c2, fcan, folac, Ialpha, M, pilhalpha, R23, R34, ZG/1, ZIK/1, ZJ/1, ZL/3, ZS/3, alpha15, f2, fr, FC3-9, K19, Mu, 01, P2, ViI, ˆ92, 121, 16-19, 9266, C16, DdVI, PST, SMB, SMP2, a1, 3, 3T+, 9/0, 11F, 50, 66F, 5845, 8893, M11, QB, ST, TW18, VK, FI, ID2, fr, f2, AE2, Ec9, C-2 f1, (syn=f-1), HR, If1, IF2, IKe, I₂-2, M13, (syn=M-13), PR64FS, SF, tf-1, X, X-2, ZG/2, ZJ2, δA B6, B7, C-1, C2, FH5, Fₒlac, fr, f2, (syn=f₂), Hgal, Iα, M, MS2, M12, (syn=M-12), pilHα, R17, (syn=R-17), SR, t, ZG/1, ZIK/1, ZJ/1, ZL/3, ZS/3, α15, μ2, (syn=μ₂) BE/1, dφ3, dφ4, dφ5, G4, G6, G13, G14, Iφ1, Iφ3, Iφ7, Iφ9, M20, St-1, (syn=St/1), (syn=ST-1), S13, (syn=S-13), U3, WA/1, WF/1, WW/1, ZD13, α3, α10, δ1, η8, o6, φA, φR, (syn=φX), (syn=φX-174), (syn=Φ174), ζ3, WS-EP13, WS-EP19
Enterococcus DF₇₈, F1, F2, 1, 2, 4, 14, 41, 867, C2, C2F E3, E62, DS96, H24, M35, P3, P9, SB101, S2, 2BII, 5, 182a, 705, 873, 881, 940, 1051, 1057, 21096C, F3, F4, VD13, 1, 200, 235, 341
Erwinia E15P, PEa7, Y46/(CE2), PEa1(h), S1, φM1, Erh1, E16P, 59, 62, 843/60
Escherichia BW73, B278, D6, D108, E, E1, E24, E41, FI-2, FI-4, FI-5, H18A, H18B, i, MM, Mu, (syn=mu), (syn=Mu1), (syn=Mu-1), (syn=MU-1), (syn=MuI), (syn=μ), O25, PhI-5, Pk, PSP3, P1, P1D, P2, P4 (defective), S1, Wφ, φK13, φR73 (defective), φ1, φ2, φ7, φ92, τ (defective), 7A, 8φ, 9φ, 15 (defective), 18, 28-1, 186, 299, NN-Escherichia (2) CFO103, HK620, J, K, K1F, m59, no. A, no. E, no. 3, no. 9, N4, sd, (syn=Sd), (syn=S_{D}), (syn=S_{d}), (syn=s_{d}), (syn=SD), (syn=CD), T3, (syn=T-3), (syn=T₃), T7 (syn=T-7), (syn=T₇), WPK, W31, Δ^{H}, φC3888, φK3, φK7, φK12, φV-1, Φ04-CF,_{—}Φ05, Φ06, Φ07, φ1, φ1.2, φ20, φ95, φ263, φ1092, φI, φII, (syn=φW), Ω8, 1, 3, 7, 8, 26, 27, 28-2, 29, 30, 31, 32, 38, 39, 42, 933W NN-Escherichia (1), Esc-7-11, AC30, CVX-5, C1, DDUP, EC1, EC2, E21, E29, F1, F26S, F27S, Hi, HK022, HK97, (syn=ΦHK97), HK139, HK253,HK256,K7,ND-1, no. D, PA-2, q, S2, T1, (syn=α), (syn=P28), (syn=T-1), (syn=T₁), T3C, T5, (syn=T-5), (syn=T₅), UC-1, w, β4, γ2, λ, (syn=Φλ), ΦD326, φγ, Φ06, Φ7, Φ10, φ80, χ, (syn=χ₁), (syn=φχ), (syn=φχ₁), 2, 4, 4A, 6, 8A 102, 150, 168, 174, 3000, AC6, AC7, AC28, AC43, AC50, AC57, AC81, AC95, HK243, K10, ZG/3A, 5, 5A, 21EL, H19-J, 933H
HaemophilusHP1, S2
Klebsiella AIO-2, Kl₄B, Kl₆B, Kl₉, (syn=Kl9), Kl₁₄, Kl₁₅, Kl₂₁, Kl₂₈, Kl₂₉, Kl₃₂, Kl₃₃, Kl₃₅, Kl₁₀₆B, Kl₁₇₁B, Kl₁₈₁B, Kl₈₃₂B, CI-1, Kl₄B, Kl₈, Kl₁₁, Kl₁₂, Kl₁₃, Kl₁₆, Kl₁₇, Kl₁₈, Kl₂₀, Kl₂₂, Kl₂₃, Kl₂₄, Kl₂₆, Kl₃₀, Kl₃₄, Kl₁₀₆B, Kl₁₆₅B, Kl₃₂₈B, KLXI, K328, P5046, 11, 380, III, IV, VII, VIII, FC3-11, Kl₂B , (syn=Kl2B), Kl₂₅, (syn=Kl25), Kl₄₂B, (syn=Kl42), (syn=Kl42B), Kl₁₈₁B, (syn=Kl181), (syn=Kl181B), Kl_{765/1}, (syn=Kl765/1), Kl₈₄₂B, (syn=Kl832B), Kl₉₃₇B, (syn=Kl937B), L1, φ28, 7, 231, 483, 490, 632 Listeria H387, 2389, 2671, 2685, 4211, A511, O1761, 4211, 4286, (syn=BO54), A005, A006, A020, A500, A502, A511, A118, A620, A640, B012, B021, B024, B025, B035, B051, B053, B054, B055, B056, B101, B 110, B545, B604, B653, C707, D441, HSO47, H1OG, H8/73, H19, H21, H43, H46, H107, H108, H110, H163/84, H312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716, NN-Listeria (15)
MicrococcusN1, N5
MycobacteriumLacticola, Leo, R1-Myb, 13, AG1, AL₁, ATCC 11759, A2, B.C₃, BG2, BK1, BK₅, butyricum, B-1, B5, B7, B30, B35, Clark, C1, C2, DNAIII, DSP₁, D4, D29, GS4E, (syn=GS₄E), GS7, (syn=GS-7), (syn=GS₇), IPα, lacticola, Legendre, Leo, L5, (syn=ΦL-5), MC-1, MC-3, MC-4, minetti, MTPH11, Mx4, MyF₃P/59a, phlei, (syn=phlei 1), phlei 4, Polonus II, rabinovitschi, smegmatis, TM4, TM9, TM10, TM20, Y7, Y10, φ630, 1B, 1F, 1H, 1/1, 67, 106, 1430, B1, (syn=Bo1), B₂₄, D, D29, F—K, F—S, HP, Polonus I, Roy, R1, (syn=R1-Myb), (syn=R₁), 11, 31, 40, 50, 103a, 103b 128, 3111-D, 3215-D, NN-Mycobacterium (1)
Mycoplasma MV-G51, NN-Mycoplasma (1), Hr1, P1
Pasteurella C-2, 32, AU, VL, TX, φPhA1, 1, 2, 10, 3/10, 4/10, 115/10, 895, 3, 22, 55, 115, 896, 994, 995, B932a, C-2, φPhA1, 32, 53, 115, 967, 1075
Proteus Pm5, 13vir, 2/44, 4/545, 6/1004, 13/807, 20/826, 57, 67b, 78, 107/69, 121, Pm1, Pm3, Pm4, Pm6, Pm7, Pm9, Pm10, Pm11, Pv2, π1, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, V1, 13/3a Clichy 12, π2600, φχ7, 1/1004, 5/742, 9, 12, 4, 22, 24/860, 2600/D52, Pm8, 24/2514
PseudomonasPhi6, Pf1, Pf2, Pf3, D3, Kf1, M6, PS4, SD1, PB-1, PP8, PS17, nKZ, nW-14, n1, 12S, AI-1, AI-2, B17, B89, CB3, Col 2, Col 11, Col 18, Col 21, C154, C163, C167, C2121, E79, F8, ga, gb, H22, K₁, M4, N₂, Nu, PB-1, (syn=PB1), pf16, PMN17, PP1, PP8, Psa1, PsP1, PsP2, PsP3, PsP4, PsP5, PS3, PS17, PTB80, PX4, PX7, PYO1, PYO2, PYO5, PYO6, PYO9, PYO10, PYO13, PYO14, PYO16, YO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, P1K, SLP1, SL2, S₂, UNL-1, wy, Ya₁, Ya₄, Ya₁₁, φBE, φCTX, φC17, φKZ, (syn=ΦKZ), φ-LT, Φmu78,_φNZ, φPLS-1, φST-1, φW-14, φ-2,1/72, 2/79, 33/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, NN-Pseudomonas (23), φ6, PP7, PRR1, 7s, NN-Pseudomonas (1), A856, B26, CI-1, CI-2, C5, D, gh-1, F116, HF, H90, K₅, K₆, K104, K109, K166, K267, N₄, N₅, O6N-25P, PE69, Pf, PPN25, PPN35, PPN89, PPN91, PP2, PP3, PP4, PP6, PP7, PP8, PP56, PP87, PP114, PP206, PP207, PP306, PP651, Psp231a, Pssy401, Pssy9220, ps₁, PTB2, PTB20, PTB42, PX1, PX3, PX10, PX12, PX14, PYO70, PYO71, R, SH6, SH133, tf, Ya₅, Ya₇, φBS, ΦKf77, φ-MC, ΦmnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, NN-Pseudomonas (5), af, A7, B3, B33, B39, BI-1, C22, D3, D37, D40, D62, D3112, F7, F10, g, gd, ge, gf, Hw12, Jb19, KF1, L°, OXN-32P, O6N-52P, PCH-1, PC13-1, PC35-1, PH2, PH51, PH93, PH132, PMW, PM13, PM57, PM61, PM62, PM63, PM69, PM105, PM113, PM681, PM682, PO4, PP1, PP4, PP5, PP64, PP65, PP66, PP71, PP86, PP88, PP92, PP401, PP711, PP891, Pssy41, Pssy42, Pssy403, Pssy404, Pssy420, Pssy923, PS4, PS-10, Pz, SD1, SL1, SL3, SL5, SM, φC5, φC11, φC11-1, φC13, φC15, φMO, φX, φ04, φ11, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351 357-1, 400-1, NN-Pseudomonas (6), G101, M6, M6a, L1, PB2, Pssy15, Pssy4210, Pssy4220, PYO12, PYO34, PYO49, PYO50, PYO51, PYO52, PYO53, PYO57, PYO59, PYO200, PX2, PX5, SL4, φ03, φ06, 1214
SalmonellaB, Beccles, CT, d, Dundee, f, Fels 2, GI, GIII, GVI, GVIII, k, K, i, j, L, O1, (syn=O-1), (syn=O₁), (syn=O-I), (syn=7), O2, O3, P3, P9a, P10, Sab3, Sab5, San15, San17, SI, Taunton, Vi1, (syn=Vi1), 9, NN-Salmonella (1), a, B.A.O.R., e, G4, GIII, L, LP7, M, MG40, N-18, PSA68, P4, P9c, P22, (syn=P₂₂), (syn=PLT22), (syn=PLT₂₂), P22a1, P22-4, P22-7, P22-11, SNT-1, SNT-2, SP6, ViIII, ViIV, ViV, ViVI, ViVII, Worksop, ε₁₅, ε₃₄, 1,37, 1(40), (syn=φ1[40]), 1,42₂, 2, 2.5, 3b, 4, 5, 6,14(18), 8, 14(6,7), 10, 27, 28B, 30, 31, 32, 33, 34, 36, 37, 39, 1412, SNT-3, 7-11, 40.3, c, C236, C557, C625, C966N, g, GV, G5, G173, h, IRA, Jersey, MB78, P22-1, P22-3, P22-12, Sab1, Sab2, Sab2, Sab4, San1, San2, San3, San4, San6, San7, San8, San9, San13, San14, San16, San18, San19, San20, San21, San22, San23, San24, San25, San26, SasL1, SasL2, SasL3, SasL4, SasL5, S1BL, SII, ViII, φ1, 1, 2, 3a, 3aI, 1010, NN-Salmonella (1), N-4, SasL6, 27
SerratioA2P, PS20, SMB3, SMP, SMP5, SM2, V40, V56, κ, ΦCP-3, ΦCP-6, 3M, 10/1a, 20A, 34CC, 34H, 38T, 345G, 345P, 501B, E20, P8, Sa1, SM4, η, ΦCP6-4, 5E, 34D, 38B, 224D1, 224D2, 2847/10b, BC, BT, CW2, CW3, CW4, CW5, L₁232, L₂232, L34, L.228, SLP, SMPA, V.43, σ, φCW1, ΦCP6-1, ΦCP6-2, ΦCP6-5, 3T, 5, 8, 9F, 10/1, 20E, 32/6, 34B, 34CT, 34P, 37, 41; 56, 56D, 56P, 60P, 61/6, 74/6, 76/4, 101/8900, 226, 227, 228, 229F, 286, 289, 290F, 512, 764a, 2847/10, 2847/10a, L.359, SMB1
ShigellaFsa, a, FS_{D2d}, (syn=D2d), (syn=W₂d), FS_{D2E}, (syn=W₂e), Fv, F6, f7.8, H-Sh, PE5, P90, SfII, Sh, SH_{III}, SH_{IV}, (syn=HIV), SH_{VI}, (syn=HVI), SHV_{VIII}, (syn=HVIII), SKγ66, (syn=gamma 66), (syn=γ66), (syn=γ66b), SK_{III}, (syn=SIIIb), (syn=III), SK_{IV}, (syn=S_{IVa}), (syn=IV), SK_{IVa}, (syn=S_{IVAa}), (syn=IVA), SK_{VI}, (syn=KVI), (syn=S_{VI}), (syn=VI), SK_{VIII}, (syn=S_{VIII}) (syn=VIII), SK_{VIIIA}, (syn=S_{VIII}A), (syn=VIIIA), ST_{VI}, ST_{IX}, ST_{XI}\pard cs1, ST_{XII}, S66, W₂, (syn=D2c), (syn=D20), φI, flV₁, 3-SO-R, 8368-SO-R, DD-2, Sf6, FS₁, (syn=F1), SF₆, (syn=F6), SG₄₂, (syn=SO-42/G), SG₃₂₀₃, (syn=SO-3203/G), SK_{F12}, (syn=SsF₁₂), (syn=F₁₂), (syn=F12), ST_{II}, (syn=1881-SO-R), γ66, (syn=gamma 66a), (syn=Ssγ66), φ2 B11, DDVII , (syn=DD7), FS_{D2b}, (syn=W₂B), FS₂, (syn=F₂), (syn=F2), FS₄, (syn=F₄), (syn=F4), FS₅, (syn=F₅), (syn=F5), FS₉, (syn=F₉), (syn=F9), F11, P2-SO—S , SG₃₆, (syn=SO-36/G), (syn=G36), SG\pard plain n₃₂₀₄, (syn=SO-3204/G), SG₃₂₄₄, (syn=SO-3244/G), SH_{I}, (syn=HI), SH_{VII}, (syn=HVII), SH_{IX}, (syn=HIX), SH_{XI}, SH_{XII}, (syn=HXII), SKI, KI, (syn=S_{I}), (syn=SsI), SKVII, (syn=KVII), (syn=S_{VII}), (syn=SsVII), SKIX, (syn=KIX), (syn=S_{IX}), (syn=SsIX), SKXII, (syn=KXII), (syn=S_{XII}), (syn=SsXII), ST_{I}, ST_{III}, ST_{IV}, ST_{VI}, ST_{VII}, S70, S206, U2-SO—S, 3210-SO—S, 3859-SO—S, 4020-SO—S, φ3, φ5, φ7, φ8, φ9, φ10, φ11, φ13, φ14, φ18, SH_{III}, (syn=HIII), SH_{XI}, (syn=HXI), SK_{XI}, (syn=KXI), (syn=S_{XI}), (syn=SsXI), (syn=XI)
   Staphylococcus 3A, B11-M15, 77, 107, 187, 2848A, Twort A, EW, K, Ph5, Ph9, Ph10, Ph13, P1, P2, P3, P4, P8, P9, P10, RG, S_{B-1}, (syn=Sb-1), S3K, φSK311, φ812, 06, 40, 58, 119, 130, 131, 200, 1623, STC1, (syn=stc1), STC2, (syn=stc2), 44AHJD, 68, AC1, AC2, A6″C″, A9″C″b⁵⁸¹, CA-1, CA-2, CA-3, CA-4, CA-5, D11, L39x35, L54a, M42, N1, N2, N3, N4, N5, N7, N8, N10, N11, N12, N13, N14, N16, Ph6, Ph12, Ph14, UC-18, U4, U15, S1, S2, S3, S4, S5, X2, Z₁, φB5-2, φD, ω, 11, (syn=φ11), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, A10, A13, b594n, D, HK2, N9, N15, P52, P87, S1, S6, Z₄, φRE, 3A, 3B, 3C, 6, 7, 16, 21, 42B, 42C, 42E, 44, 47, 47A, 47C, 51, 54, 54x1, 70, 73, 75, 78, 81, 82, 88, 93, 94, 101, 105, 110, 115, 129/16, 174, 594n, 1363/14, 2460, NN-Staphylococcus (1)
StreptococcusA25, A25 PE1, A25 VD13, A25 omega8, EJ-1, NN-Streptococcus (1), a, Cl, F_{LO}Ths, H39, Cp-1, Cp-5, Cp-7, Cp-9, Cp-10, AT298, A5, a10/J1, a10/J2, a10/J5, a10/J9, A25, BT11, b6, CA1, c20-1, c20-2, DP-1, Dp-4, DT1, ET42, e10, F_{A}101, F_{E}Ths, F_{K}, F_{KK}101, F_{KL}10, F_{KP}74, F_{K}11, F_{LO}Ths, F_{Y}101, f1, F₁₀, F₂₀140/76, g, GT-234, HB3, (syn=HB-3), HB-623, HB-746, M102, O1205, φO1205, PST, P0, P1, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, Sfi11, (syn=SFi11), (syn=φSFi11), (syn=ΦSfi11), (syn=φSfi11), sfi19, (syn=SFi19), (syn=φSFi19), (syn=φSfi19), Sfi21, (syn=SFi21), (syn=φSFi21), (syn=pSfi21), ST_{G}, STX, st2, ST₂, ST₄, S3, (syn=φS3), s265, Φ17, φ42, Φ57, φ80, φ81, φ82, φ83, φ84, φ85, φ86, φ87, φ88, φ89, φ90, φ91, φ92, φ93, φ94, φ95, φ96, φ97, φ98, φ99, φ100, φ101, φ102, φ227, φ7201, ω1,_{—}ω2, ω3, ω4, ω5, ω6, ω8, ω10, 1, 6, 9, 10F, 12/12, 14, 17SR, 19S, 24, 50/33, 50/34, 55/14, 55/15, 70/35, 70/36, 71/ST15, 71/45, 71/46, 74F, 79/37, 79/38, 80/J4, 80/J9, 80/ST16, 80/15, 80/47, 80/48, 101, 103/39, 103/40, 121/41, 121/42, 123/43, 123/44, 124/44, 337/ST17, NN-Streptococcus (34)
StreptomycesSK1, type IV, CRK, SLE111, Φ17, (syn=φ17), (syn=2a), 1, 9, 14, 24 A, AP-3, AP-2863, Bα, B-I, B-II, CPC, CPT, CT, CTK, CWK, ES, FP22, FP43, K, MSP4, MSP7, MSP10, MSP11, MSP15, MSP16, MSP17, MSP18, MSP19, MVP4, MVP5, P8, P9, P13, P23, RP2, RP3, RP10, R₁, R4, SAP1, SAP2, SAP3, SAt1, SA6, SA7, SC1, SH10, SL1, SV2, TG1, type Ia, type II, type V, VC11, VP1, VP5, VP7, VP11, VWB, VW3, WSP3, φA1, φA2, φA3, φA4, φA5, φA6,_φA7, φA8, φA9, φBP1, φBP2, φC31, (syn=φc31), (syn=φ31C), (syn=C31), φC43, φHAU3, φSF1, φSPK1, 4, 5a, 5b, 8, 10, 12b, 13, 17, 19, 22, 23, 25, 506, NN-Streptomyces (3), Mex, MLSP1, MLSP2, MLSP3, MSP1, MSP2, MSP3, MSP5, MSP8, MSP9, MSP12, MSP13, MSP14, R₂, SA1, SA2, SA3, SA4, SA5, type I, type Ia, (syn=35/35), type III, type IV, WSP1, WSP4, WSP5, 2b, 4, 15, (syn=C), 26, 8238
VibrioOXN-52P, VP-3, VP5, VP11, alpha3alpha, IV, kappa, 06N-22-P, VP1, x29, II, nt-1, CP-T1, ET25, kappa, K139, Labol, OXN-69P, OXN-86, 06N-21P, PB-1, P147, rp-1, SE3, VA-1, (syn=VcA-1), VcA-2, VcA-3, VP1, VP2, VP4, VP7, VP8, VP9, VP10, VP17, VP18, VP19, X29, (syn=29 d'Hérelle), β, ΦHAWI-1, ΦHAWI-2, ΦHAWI-3,_ΦHAWI-4, ΦHAWI-5,_ΦHAWI-6, ΦHAWI-7, ΦHAWI-8, ΦHAWI-9, ΦHAWI-10, ΦHC1-1, ΦHC1-2, ΦHC1-3, ΦHC1-4,_ΦHC2-1, ΦHC2-2, ΦHC2-3,_ΦHC2-4,_ΦHC3-1, ΦHC3-2, ΦHC3-3,_ΦHD1S-1,_ΦHD1S-2, ΦHD2S-1, ΦHD2S-2, ΦHD2S-3,_ΦHD2S-4, ΦHD2S-5, ΦHDO-1, ΦHDO-2,_ΦHDO-3, ΦHDO-4, ΦHDO-5, ΦHDO-6,_KL-33, ΦKL-34,_ΦKL-35, ΦKL-36, ΦKWH-2, ΦKWH-3, ΦKWH-4, ΦMARQ-1, ΦMARQ-2,_ΦMARQ-3, ΦMOAT-1, ΦO139, ΦPEL1A-1, ΦPEL1A-2, ΦPEL8A-1, ΦPEL8A-2, ΦPEL8A-3,_ΦPEL8C-1, ΦPEL8C-2, ΦPEL13A-1, ΦPEL13B-1, ΦPEL13B-2, ΦPEL13B-3, ΦPEL13B-4,_ΦPEL13B-5, ΦPEL13B-6, ΦPEL13B-7, ΦPEL13B-8, ΦPEL13B-9, ΦPEL13B-10, ΦVP143, ΦVP253, Φ16, φ38, 1-11, 5, 13, 14, 16, 24, 32 493, 6214, 7050, 7227, II, (syn=group II), (syn=_{—}φ2), V, VIII, NN-Vibrio (13), CTXΦ, fs, (syn=fs1), fs2, lvpf5, Vf12, Vf33, VPIΦ, VSK, v6, 493, e1, e2, e3, e4, e5, FK, G, J, K, nt-6, N1, N2, N3, N4, N5, O6N-34P, OXN-72P, OXN-85P, OXN-100P, P, Ph-1, PL163/10, Q, S, T, φ92, 1-9, 37, 51, 57, 70A-8, 72A-4, 72A-10, 110A-4, 333, 4996, I, (syn=group I), III, (syn=group III), VI, (syn=A-Saratov), VII, IX, X, NN-Vibrio (6), pA1, 77-8, 70A-2, 71A-6, 72A-5, 72A-8, 108A-10, 109A-6, 109A-8, 110A-1, 110A-5, 110A-7, hv-1, OXN-52P, P13, P38, P53, P65, P108, P111, TP1, VP3, VP6, VP12, VP13, 70A-3, 70A-4, 70A-10, 72A-1, 108A-3, 109-B1, 110A-2, 149, (syn=φ149), IV, (syn=group IV), NN-Vibrio (22), VP5, VP11, VP15, VP16, α1, α2, α3a, α3b, 353B, NN-Vibrio (7)
XanthomonasCf, Cf1t, Xf, Xf2, XP5, HP1, OX1, (syn=XO1), OX2, SBX-1, XCVP, XTP1, Cf, Cf1t, Xf, (syn=xf) Xf2, φLf, φXo, φXv, RR68, A342, HXX, PG60, P1-3a, P6, XO3, XO4, XO5, 20, 22, NN-Xanthomonas (1), XP12, (syn=XP-12), (syn=Xp12), φPS, φRS, φSD, φSL, φ56, φ112, 1
YersiniaH, H-1, H-2, H-3, H-4, Lucas 110, Lucas 404, Lucas 303, YerA3, YerA7, YerA20, YerA41, 3/M64-76, 5/G394-76, 6/C753-76, 8/C239-76, 9/F18167, 1701, 1710, D'Hérelle, EV, H, Kotljarova, PTB, R, Y, YerA41, φYerO3-12, 3, 4/C1324-76, 7/F783-76, 903, 1/M6176, Yer2AT,
   (Phage names courtesy of Hans-Wolfgang Ackermann & Stephen Tobias Abedon (2001) Bacteriophage Names, 2000. The Bacteriophage Ecology Group,)

There are numerous other phages infecting these and other bacteria. The bacteriophages are normally grouped into family, genus and species, including but not limited to the following genera: Bdellomicrovirus, Spiromicrovirus, Microvirus, Microvirus, Levivirus, Allolevivirus. It should be noted that the compositions of embodiments of the disclosure contain phage peptides and peptide fragments thereof as well as, or instead of, phage proteins.

Prophylactic Methods

There are several different bacteria that can cause mastitis. Streptococcus aglactiae, Staphylococcus aureus, and Mycobacterium species are found in the mammary gland, which serves as the principal site of persistence or reservoir (Thomson's Special Veterinary Pathology, Edition 3, 2001, p. 627). Some coliform organisms use the environment as the reservoir. Coliform bacteria that may cause mastitis include E. coli, Enterobacter aerogenes, and Klebsiella pneumoniae. Actinomyces pyogenes causes a mastitis in lactating, nonlactating, and even immature bovine mammary glands, which may sometimes be characterized by abscesses in the tissue about the large and small lactiferous ducts. (Thomson Special Veterinary Pathology, p. 631).

Additionally, there are some bacteria that use either the environment or the mammary gland as the reservoir. This group includes Streptococcus uberis and Streptococcus dysgalactiae.

In addition to the bacteria listed above, tuberculosis mastitis may be caused by Mycobacterium bovis, arriving in the mammary gland hematogenously from organs with previously established tubercles.

Of course, cows are not the only animals to suffer from mastitis. Pasteurella haemolytica can cause mastitis in lactating sheep, and sometimes also cause rhinits and pneumonia in their lambs.

The method for prophylactically or therapeutically treating these bacterial infections causing mastitis comprises treating the infection with a therapeutic agent comprising an effective amount of at least one lytic enzyme genetically coded for a bacteriophage specific for digesting the cell wall of a specific bacteria. The lytic enzyme is preferably in an environment having a pH which allows for activity of said lytic enzyme. The lytic enzyme may be “unaltered,” chimeric, shuffled or any combination thereof. Additionally, a holin protein may be included in a composition containing the lytic enzyme(s).

While an “unmodified” or “unaltered” phage associated lytic enzyme may be used for treatment of bacteria that cause respiratory infections, it may be preferred that a shuffled or chimeric lytic enzyme be used, possibly with a holin protein.

There are a number of ways to apply the lytic enzyme.

In those instances where it is desirable to prevent mastitis where the bacteria causing the mastitis is in the environment, the enzyme may be put in a liquid carrier and sprayed in the general area of the pen of the nursing animal.

Prior to, or at the time the lysin enzyme is put in the carrier system or oral delivery mode, it is preferred that the enzyme be in a stabilizing buffer environment for maintaining a pH range of between about 4.0 and about 9.0, more preferably between about 5.5 and about 7.5 and most preferably at about 6.1.

The stabilizing buffer should allow for the optimum activity of the lysin enzyme. The buffer may be a reducing reagent, such as dithiothreitol. The stabilizing buffer may also be or include a metal chelating reagent, such as ethylenediaminetetraacetic acid disodium salt, or it may also contain a phosphate or citrate-phosphate buffer. Other appropriate buffers may be used.

The solutions in which the enzyme is placed may be a water solution, oil based solution, saline solution, or any other means for carrying the enzyme.

These enzymes can also be administered nutritionally. Each and every one of these lytic enzymes, chimeric lytic enzymes, shuffled lytic enzymes, and combinations of enzymes can be included in a food product, be it liquid or be it foodstuff. The enzymes can be enterically coated or they can be included in a liposome.

When an animal has potentially been exposed to any of these or other pathogens in water or food, treatment may begin to prevent the growth and spread of the bacteria throughout the body, using any of the methods described above.

Prior to treating the infected animal(s), it may be desirable to test and determine which bacteria is causing the mastitis. It should be noted that the therapeutic agent, which is comprised of the lytic enzyme(s) and the carrier, may in fact contain more than one enzyme for treating a specific lytic enzyme, and more than one enzyme may be in the therapeutic agent, with each enzyme being specific for a specific bacteria.

There are a number of ways in which the enzyme may be given or applied to the animal to cure the mastitis. The three principle approaches to treating mastitis using lytic enzymes are parenteral delivery, oral delivery, and topical delivery. However, an alternative and preferred method of treating mastitis in animals is to inject the lytic enzyme through the opening of the teat into the udder.

Each of these techniques will be explored below.

A number of different methods may be used to introduce the lytic enzyme(s) parenterally. These methods include introducing the lytic enzyme intravenously, intramuscularly, subcutaneously, subdermally, and intrathecally. The therapeutic agent should comprise the appropriate and effective amount of the lytic enzyme(s) (holin lytic enzyme, chimeric lytic enzyme and/or shuffled lytic enzyme) in combination with a carrier comprising distilled water, a saline solution, albumin, a serum, or any combination thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils and ethyl oleate are also useful herein, although are usually not recommended for intravenous use. A straight intravenous solution with the enzyme in the appropriate solutions and buffers is best.

The carrier suitably contains minor amounts of additives such as substances that enhance isotonicity and chemical stability. Such materials are nontoxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% and more, but preferably about 20%.

The carrier vehicle may also include Ringer's solution, a buffered solution, and dextrose solution, particularly when an intravenous solution is prepared.

The chimeric and/or shuffled lytic enzymes may be used in combination with other chimeric and shuffled lytic enzymes, holin proteins, other lytic enzymes, and other phage associated lytic enzymes which have not been modified or which are not “recombinant.”

While the most serious infectious cases should be treated in a veterinary clinic or hospital, it is possible for the animal to receive an appropriate enzyme solution by a portable pump that may be either a mechanical or electro-mechanical pump. In some cases, the patients may receive daily or two or more injections a day of the enzyme solution. Additionally, while intravenous is recommended for many animals, there may be subcutaneous, intramuscular and other forms of parental administration in other cases where the bacteria has infected other specific parts of the body.

It should also be noted that the lytic enzyme can be administered parenterally by means of a continuous drip, or by one or more daily injections.

Additionally, the enzyme may possibly be delivered orally, in the form of pills, with the enzyme preferably in a micelle or liposome. Additionally, the liposomes and micelles may be “nano” sized. Additionally, the lytic enzyme may be administered in a rectal suppository for absorption, or by a syrup. Dosage rates can be in the range of 1,000 to 100,000 units/ml, although dosages can be as high as 5,000,000 to 10,000,000 units/ml.

Carriers for animal uses could be also be in the form of a food additive, the additive (being the enzyme and possibly a carrier) being added to hay, grasses, fruits, water sources, pills, candies, dry and wet and dry grasses. Doses may vary, depending on the animal, on the animal's size, and the severity of the illness.

The therapeutic agent may also be topically applied to the teats of the animal so as to treat external infections, thereby limiting the spread of the bacteria to other animals and help stop reinfection of the mammary gland itself. The compositions for treating mastitis bacteria on the skin of the teat(s) comprises administering a therapeutic agent having an effective amount of at least one lytic enzyme produced by a bacteria infected with a bacteriophage specific for the bacteria and a carrier for delivering at least one lytic enzyme to the wounded skin. The composition may be either supplemented by chimeric and/or shuffled lytic enzymes, or may themselves be chimeric and/or shuffled lytic enzymes. Similarly, a holin protein may be included, which may also be a chimeric and/or shuffled lytic protein. The mode of application for the lytic enzyme includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. A mode of delivery of the carrier containing the therapeutic agent includes but is not limited to a smear, spray, a time-release patch, a liquid absorbed wipe, and combinations thereof. The lytic enzyme may be applied to a bandage either directly or in one of the other carriers. The bandages may be damp or dry, wherein the enzyme is in a lyophilized form on the bandage.

The carriers of the compositions of the present disclosure may comprise semisolid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament.

Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B. F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Preferably, the gelling agent comprises between about 0.2% to about 4% by weight of the composition. More particularly, the preferred compositional weight percent range for “CARBOPOL®” is between about 0.5% to about 2%, while the preferred weight percent range for “NATROSOL®” and “KLUCEL®” is between about 0.5% to about 4%. The preferred compositional weight percent range for both “HYPAN®” and “STABILEZE®” is between about 0.5% to about 4%. CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

Preservatives may also be used in this disclosure and preferably comprise about 0.05% to 0.5% by weight of the total composition. The use of preservatives assures that if the product is microbially contaminated, the formulation will prevent or diminish microorganism growth. Some preservatives useful in this disclosure include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

Titanium dioxide may be used as a sunscreen to serve as prophylaxis against photosensitization. Alternative sun screens include methyl cinnamate. Moreover, BHA may be used as an antioxidant, as well as to protect ethoxydiglycol and/or dapsone from discoloration due to oxidation. An alternate antioxidant is BHT.

Pharmaceuticals for use in all embodiments of the disclosure include antimicrobial agents, anti-inflammatory agents, antiviral agents, local anesthetic agents, corticosteroids, destructive therapy agents, antifungals, and antiandrogens. In the treatment of acne, active pharmaceuticals that may be used include antimicrobial agents, especially those having anti-inflammatory properties such as dapsone, erythromycin, minocycline, tetracycline, clindamycin, and other antimicrobials. The preferred weight percentages for the antimicrobials are 0.5% to 10%. Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is about 0.025% to 5% by weight of the total composition. Anesthetics such as benzocaine may also be used at a preferred concentration of about 2% to 25% by weight.

Corticosteroids that may be used include betamethasone dipropionate, fluocinolone actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide at concentrations of about 0.01% to about 1.0% by weight. Preferred concentrations for corticosteroids such as hydrocortisone or methylprednisolone acetate are from about 0.2% to about 5.0% by weight.

Destructive therapy agents such as salicylic acid or lactic acid may also be used. A concentration of about 2% to about 40% by weight is preferred. Cantharidin is preferably utilized in a concentration of about 5% to about 30% by weight. Typical antifungals that may be used in this disclosure and their preferred weight concentrations include: oxiconazole nitrate (0.1% to 5.0%), ciclopirox olamine (0.1% to 5.0%), ketoconazole (0.1% to 5.0%), miconazole nitrate (0.1% to 5.0%), and butoconazole nitrate (0.1% to 5.0%).

In one embodiment, the disclosure comprises a dermatological composition having about 0.5% to 10% carbomer and about 0.5% to 10% of a pharmaceutical that exists in both a dissolved state and a micro particulate state. Addition of an amine base, potassium, hydroxide solution, or sodium hydroxide solution completes the formation ofthe gel. More particularly, the pharmaceutical may include dapsone, an antimicrobial agent having anti-inflammatory properties. A preferred ratio of micro particulate to dissolved dapsone is five or less.

In another embodiment, a composition comprises about 1% carbomer, about 80-90% water, about 10% ethoxydiglycol, about 0.2% methylparaben, about 0.3% to 3.0% dapsone including both micro particulate dapsone and dissolved dapsone, and about 2% caustic material. More particularly, the carbomer may include “CARBOPOL® 980“and the caustic material may include sodium hydroxide solution.

In a preferred embodiment, the composition comprises dapsone and ethoxydiglycol, which allows for an optimized ratio of micro particulate drug to dissolved drug. This ratio determines the amount of drug delivered, compared to the amount of drug retained in or above the stratum comeum to function in the supracomeum domain. The system of dapsone and ethoxydiglycol may include purified water combined with “CARBOPOL®” gelling polymer, methylparaben, propylparaben, titanium dioxide, BHA, and a caustic material to neutralize the “CARBOPOL.®”

Any of the carriers for the lytic enzyme may be manufactured by conventional means. However, if alcohol is used in the carrier, the enzyme should be in a micelle, liposome, or a “reverse” liposome, or reverse micelle, to prevent denaturing of the enzyme. Similarly, when the lytic enzyme is being placed in the carrier, and the carrier is, or has been heated, such placement should be made after the carrier has cooled somewhat, to avoid heat denaturation of the enzyme. In a preferred embodiment of the disclosure, the carrier is sterile.

As noted above, the preferred treatment of mastitis in animals is by injecting the lytic enzyme through the opening of the teat into the udder. This method has the advantage of localizing the treatment while at the same time providing for less diffusion of the lytic enzyme throughout the body, allowing for a very high concentration of lytic enzyme at the site of the bacterial infection of the mammary gland.

While a syringe may be or some sort of injection device may be used to deliver the therapeutic agent, the carriers for either the parenteral or the topical method of delivery may be used. Various medicaments of both the liquid topical and/or parenteral carriers may be included in the therapeutic agent for introduction into the teat. More generally, the various medicaments, buffers, and other components of the topical and parenteral carriers may be included in the therapeutic agent to be introduced through the teat. For example, anti-inflammatories, local anesthetics, anti-oxidants, a variety of cortisones, and numerous other healants disclosed above may be included in the therapeutic agent. It is also preferred that the carrier is a liquid, but no other possible forms of the carrier should be excluded, including those carriers for topical use.

The effective dosage rates or amounts of the lytic enzyme to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1000 units/ml to about 100,000 units/ml, and most preferably from about 10,000 to 100,000 units/ml. The amount of active units per ml may, in some circumstances, be as high as 5-10 million units/ml. The number of active units and the duration of time of exposure depends on the nature of the infection, and the amount of contact the carrier allows the lytic enzyme(s) to have. This dosage rate may be used or found in any of the means of delivery. It is to be remembered that the enzyme works best when in a fluid environment. Hence, effectiveness of the enzyme(s) is in part related to the amount of moisture trapped by the carrier. In another preferred embodiment, a mild surfactant is present in an amount effective to potentiate the therapeutic effect of the lytic enzyme. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, e.g., fatty acids, glycerides, monoglycerides, deoxycholate and esters of deoxycholate.

More than one lytic enzyme may be introduced into the infected body at a time.

If there has been either exposure or potential exposure of the animal to a bacteria, prophylactic treatment should begin as soon as possible after exposure. Prophylactic treatment can be the same treatment as that of therapeutic treatment.

The lytic enzyme may be produced by standard techniques, such as by incorporating the genetic coding for the enzyme in a vector, which is introduced into a bacteria which can produce the enzyme without itself being lysed. Any other technique may be used for production. This applies to any and all uses of the enzyme in treating any illness. Fermentation type factories may be used to produce the enzymes en mass. These techniques are well known in the art.

It is preferred that the enzyme should be incorporated into a carrier which does not contain alcohol, and which has been cooled to a temperature that will not cause the permanent denaturing of or damage to the enzyme. The enzyme may be incorporated in a lyophilized state, before being incorporated into the carrier. Additionally the enzyme may be in a micelle, reverse micelle, liposome, or some other chemical structure which would have the advantage of protecting the lytic enzyme and extending its useful life.

Furthermore, in case of systemic infection, the enzyme can be used in combination or in conjunction with a very modified antibiotic treatment. The neutralization of the localized infection by the enzyme would thus demonstrably reduce the amount of antibiotics needed for treatment.

The enzyme placed in the composition or carrier should be in an environment having a pH which allows for activity of the lytic enzyme. To this end, the pH of the composition is preferably kept in a range of between about 2 and about 11. and even more preferably at a pH range of between 5.5 and 7.5. As described above with the other lytic enzyme, the pH can be moderated by the use of a buffer. The buffer may contain a reducing agent, and more specifically dithiothreitol. The buffer may also be a metal chelating reagent, such as ethylenediaminetetracetic disodium salt or the buffer may contain a citrate-phosphate buffer. As with all compositions described in this patent, the composition may further include a bactericidal or bacteriostatic agent as a preservative.

It is to be remembered that each bacteria is susceptible to numerous bacteriophages, each coding for a lytic enzyme that can lyse that specific bacteria. Any individual, variety, or combination of lytic enzymes, chimeric lytic enzymes, and/or shuffled lytic enzymes may be used.

Additionally, further techniques may be used to prevent the spread of the listed bacteria either into the food chain or onto the food directly.

In addition to the use of modified and unmodified lytic enzymes, Similarly, a holin protein may be included in the therapeutic agent, with the holin protein being either chimeric and/or shuffled.

Enzyme Delivery

It is expected that the enzymes will only have to be in the body a short time before they destroy the targeted bacteria. However, it may be necessary to make certain modifications to the bacteria, or to put the bacteria in a protected environment, to aid in their delivery and destruction of the bacteria.

In one preferred embodiment of the disclosure, the enzyme may be pegylated. For example, one or more activated poly(ethylene glycol) (PEG) derivatives, preferably from Shearwater Polymers, Inc., is attached to the enzyme. More specifically, PEG is a neutral, water-soluble, non-toxic polymer. The lack of toxicity from pegylation is reflected in the fact that PEG is one of the few synthetic polymers approved for internal use by the FDA, appearing in food, cosmetics, personal care products and pharmaceuticals. The true nature of PEG, however, is revealed by its behavior when dissolved in water. In an aqueous

By using PEGs, there is reduced immunogenicity and proteolysis. Carbohydrate and peptide receptor clearance mechanisms are “fooled” by PEG's “cloaking” ability. Less frequent dosing is required due to greatly increased body residence time. There is also improved efficacy due to increased concentration and longer dwell time at the site of action.

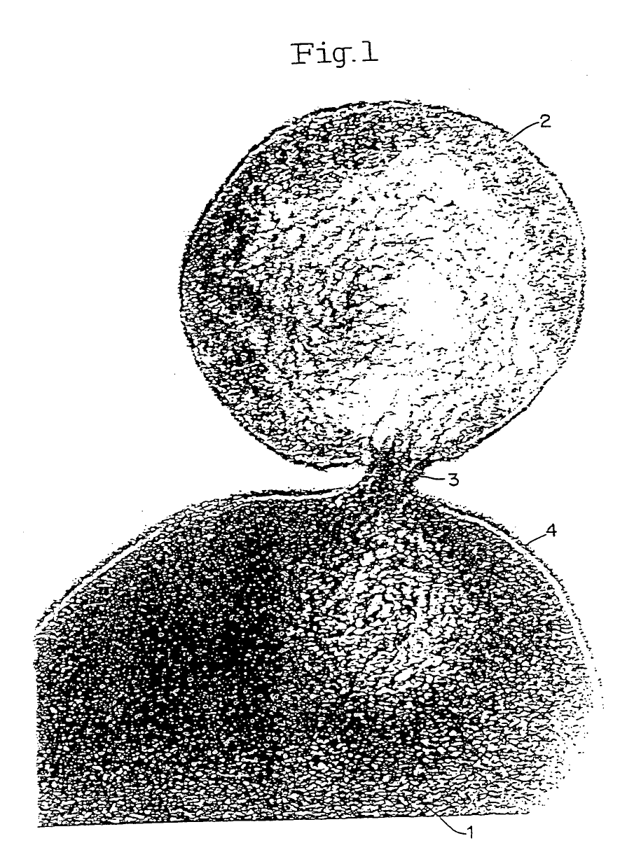

The use of lytic enzymes, including but not limited to holin lytic enzymes, chimeric lytic enzymes, shuffled lytic enzymes, and combinations thereof, rapidly lyse the bacterial cell. The thin section electron micrograph of FIG. 1 shows the results of a group A streptococci 1 (not a cause of mastitis) treated for 15 seconds with lysin. The micrograph (25,000× magnification) shows the cell contents 2 pouring out through a hole 3 created in the cell wall 4 by the lysin enzyme.

As noted above, the use of the holin lytic enzyme, the chimeric lytic enzyme, and/or the shuffled lytic enzyme, may be accompanied by the use of a “natural” lytic enzyme, which has not been modified by the methods cited in U.S. Pat. No. 6,132,970, or by similar state of the art methods. Similarly, the natural proteins or lytic enzyme may be used without the chimeric or shuffled lytic enzymes. The phage associated lytic enzyme (not limited by the bacterial lytic enzymes discussed above) may be prepared as shown in the following example:

### EXAMPLE 1

### Harvesting Phage Associated Lytic Enzyme

Group C streptococcal strain 26RP66 (ATCC #21597) or any other group C streptococcal strain is grown in Todd Hewitt medium at 37 degrees C. to an OD of 0.23 at 650 nm in an 18 mm tube. Group C bacteriophage (C1) (ATCC #21597-B1) at a titer of 5,000,000 is added at a ratio of 1 part phage to 4 parts cells. The mixture is allowed to remain at 37 degrees C. for 18 min at which time the infected cells are poured over ice cubes to reduce the temperature of the solution to below 15 degrees C. The infected cells are then harvested in a refrigerated centrifuge and suspended in 1/300th of the original volume in 0.1 M phosphate buffer, pH 6.1 containing 5 mm dithiothreitol and 10 ug of DNAase. The cells will lyse releasing phage and the lysin enzyme. After centrifugation at 100,000 g for 5 hrs to remove most of the cell debris and phage, the enzyme solution is aliquoted and tested for its ability to lyse Group A Streptococci.

The number of units/ml in a lot of enzyme is determined to be the reciprocal of the highest dilution of enzyme required to reduce the OD650 of a suspension of group A streptococci at an OD of 0.3 to 0.1 5 in 15 minutes. In a typical preparation of enzyme 400,000 to 4,000,000 units are produced in a single 12 liter batch.

Use of the enzyme in an immunodiagnostic assay requires a minimum number of units of lysin enzyme per test depending on the incubation times required. The enzyme is diluted in a stabilizing buffer maintaining the appropriate conditions for stability and maximum enzymatic activity, inhibiting nonspecific reactions, and in some configurations contains specific antibodies to the Group A carbohydrate. The preferred embodiment is to use a lyophilized reagent which can be reconstituted with water. The stabilizing buffer can comprise a reducing reagent, which can be dithiothreitol in a concentration from 0.001M to 1.0M, preferably 0.005M. The stabilizing buffer can comprise one or more immunoglobulin or immunoglobulin fragments in a concentration of 0.001 percent to 10 percent, preferably 0.1 percent. The stabilizing buffer can comprise a citrate-phosphate buffer in a concentration from 0.001M to 1.0M, preferably 0.05M. The stabilizing buffer can have a pH value in the range from 5.0 to 9.0. The stabilizing buffer can comprise a bacteriacidal or bacteriostatic reagent as a preservative. Such preservative can be sodium azide in a concentration from 0.001 percent to 0.1 percent, preferably 0.02 percent.

The preparation of phage stocks for lysin production is the same procedure described above for the infection of group C streptococcus by phage in the preparation of the lysin enzyme. However, instead of pouring the infected cells over ice, incubation at 37 degrees C. is continued for a total of 1 hour to allow lysis and release of the phage and the enzyme in the total volume. In order for the phage to be used for subsequent lysin production the residual enzyme must be inactivated or removed to prevent lysis from without of the group C cells rather than phage infection.

In all of the uses for the enzyme, the form of the enzyme may be “natural,” formed by recombinant or “genetically engineered” means, and may be a shuffled, chimeric or otherwise altered enzyme. A holin protein may be used in any of the illnesses discussed, and more than one enzyme may be used in each composition.

Each publication cited herein is incorporated by reference in its entirety. Any part of Harrison's Principles of Internal Medicine, 15^{th} Edition and Robins Pathologic Basis of Infectious Diseases from which much of the background information was obtained which was not properly cited is purely an oversight.

Many modifications and variations of the present disclosure are possible in light of the above teachings. It is, therefore, to be understood within the scope of the appended claims the disclosure may be protected otherwise than as specifically described.

What we claim is:

1) A method of treating mastitis in animals, said method comprising:
administering an effective amount of a therapeutic agent to a mammary gland, said therapeutic agent comprising:
a) an effective amount of at least one lytic enzyme genetically coded for by a specific bacteriophage specific for a bacteria infecting said mammary glands animals, wherein the bacteria to be treated is selected from the group consisting of Streptococcus aglactiae, Staphylococcus aureus, Mycobacterium sp, E. coli, Enterobacter aerogenes, Klebsiella pneumoniae, Actinomyces pyogenes Streptococcus uberis and Streptococcus dysgalactiae, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mammary gland.

2) The method according to claim 1, wherein said bacteria being treated is Streptococcus aglactiae.

3) The method according to claim 1, wherein said bacteria being treated is Staphylococcus aureus.

4) The method according to claim 1, wherein said bacteria being treated is Mycobacterium sp.

5) The method according to claim 1, wherein said bacteria being treated is E. coli.

6) The method according to claim 1, wherein said bacteria being treated is Enterobacter aerogenes.

7) The method according to claim 1, wherein said bacteria being treated is Klebsiella pneumoniae.

8) The method according to claim 1, wherein said bacteria being treated is Actinomyces pyogenes

9) The method according to claim 1, wherein said bacteria treated is Streptococcus uberis

10) The method according to claim 1, wherein said bacteria treated is Streptococcus dysgalactiae.

11) The method according to claim 1, wherein said pharmaceutical carrier is a parenteral carrier.

12) The method according to claim 1, wherein said pharmaceutical carrier is a topical carrier.

13) The method according to claim 12, wherein said therapeutic agent is applied inside a teat of said mammal.

14) A composition for treating mastitis in animals, said mastitis comprising:
an effective amount of a therapeutic agent to a mammary gland, said therapeutic agent comprising:
a) an effective amount of at least one lytic enzyme genetically coded for by a specific bacteriophage specific for a bacteria infecting said mammary glands animals, wherein the bacteria to be treated is selected from the group consisting of Streptococcus aglactiae, Staphylococcus aureus, Mycobacterium sp, E. coli, Enterobacter aerogenes, Klebsiella pneumoniae, Actinomyces pyogenes Streptococcus uberis and Streptococcus dysgalactiae, wherein said at least one said lytic enzyme is specific for and has the ability to digest a cell wall of one of said bacteria and is coded for by the same said bacteriophage capable of infecting said bacteria being digested; and
b) a pharmaceutically acceptable carrier for delivering said at least one lytic enzyme to the mammary gland.

15) The composition according to claim 14, wherein said bacteria being treated is Streptococcus aglactiae.

16) The composition according to claim 14, wherein said bacteria being treated is Staphylococcus aureus.

17) The composition according to claim 14, wherein said bacteria being treated is Mycobacterium sp.

18) The composition according to claim 14, wherein said bacteria being treated is E. coli.

19) The composition according to claim 14, wherein said bacteria being treated is Enterobacter aerogenes.

20) The composition according to claim 14, wherein said bacteria being treated is Klebsiella pneumoniae.

21) The composition according to claim 14, wherein said bacteria being treated is Actinomyces pyogenes

22) The composition according to claim 14, wherein said bacteria treated is Streptococcus uberis

23) The composition according to claim 14, wherein said bacteria treated is Streptococcus dysgalactiae.

24) The composition according to claim 14, wherein said pharmaceutical carrier is a parenteral carrier.

25) The composition according to claim 1, wherein said pharmaceutical carrier is a topical carrier.

26) The composition according to claim 12, wherein said therapeutic agent is applied inside a teat of said mammal.
